(19) United States
(12) Patent Application Publication
(10) Pub. No.: US 2024/0260832 A1
(43) Pub. Date: Aug. 8, 2024

(54) IMAGING SYSTEM, PROCESSING DEVICE, AND METHOD TO BE PERFORMED BY COMPUTER IN IMAGING SYSTEM

(71) Applicant: Panasonic Intellectual Property Management Co., Ltd., Osaka (JP)

(72) Inventor: TAKAMASA ANDO, Osaka (JP)

(21) Appl. No.: 18/624,249

(22) Filed: Apr. 2, 2024

Related U.S. Application Data
(63) Continuation of application No. PCT/JP2022/035983, filed on Sep. 27, 2022.

(30) Foreign Application Priority Data
Nov. 5, 2021 (JP) 2021-181379

(51) Int. Cl. A61B 5/00; A61B 5/026; H04N 23/611; H04N 23/695

(52) U.S. Cl. CPC A61B 5/0042; A61B 5/0261; H04N 23/611; H04N 23/695; A61B 2562/0233; A61B 2576/026

(57) ABSTRACT

An imaging system includes: a first imaging device having a first field of view; a second imaging device having a second field of view narrower than the first field of view; and a motor-driven device capable of changing an orientation of the second imaging device, in which the first imaging device images a living body and generates first image data, the second imaging device images a target portion of the living body and generates second image data, the second image data is sent to a processing device that generates, based on the second image data, data indicating biological information of the target portion, and the motor-driven device changes the orientation of the second imaging device, based on a position of the living body in an image based on the first image data and maintains a state in which the target portion is included in the second field of view.

### BACKGROUND

### 1. Technical Field

The present disclosure relates to an imaging system, a processing device, and a method to be performed by a computer in an imaging system.

### 2. Description of the Related Art

Reflected light produced by irradiating a target portion of a living body with light includes a component from the surface and that from inside the target portion. When such reflected light is detected, biological information of the target portion, such as surface information and/or internal information, can be obtained. Japanese Unexamined Patent Application Publication No. 11-164826 and Japanese Unexamined Patent Application Publication No. 4-189349 disclose apparatuses that obtain internal information of a target portion.

### SUMMARY

In an environment in which a living body moves, biological information of a target portion might not be stably obtained. One non-limiting and exemplary embodiment provides an imaging system capable of stably obtaining, in an environment in which a living body moves, biological information of a target portion of the living body in a noncontact manner.

In one general aspect, the techniques disclosed here feature an imaging system including: a first imaging device that has a first field of view; a second imaging device that has a second field of view narrower than the first field of view; and a motor-driven device that is capable of changing an orientation of the second imaging device, in which the first imaging device images a living body and generates first image data, the second imaging device images a target portion of the living body and generates second image data, the second image data is sent to a processing device that generates, on the basis of the second image data, data indicating biological information of the target portion, and the motor-driven device changes the orientation of the second imaging device on the basis of a position of the living body in an image based on the first image data and maintains a state in which the target portion is included in the second field of view.

According to the techniques of the present disclosure, an imaging system capable of stably obtaining, in an environment in which a living body moves, biological information of a target portion of the living body in a noncontact manner can be implemented.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a block diagram schematically illustrating a configuration of an imaging system according to an exemplary embodiment of the present disclosure;

FIG. 1B is a diagram schematically illustrating a first light source, a second light source, a second imaging device, and a control circuit and a signal processing circuit included in a processing device, which are included in the imaging system in FIG. 1A;

FIG. 2 is a flowchart schematically illustrating an example correction operation performed by the processing device when a living body moves;

FIG. 3A is a diagram for explaining operations of a motor-driven device;

FIG. 3B is a diagram for explaining operations of the motor-driven device;

FIG. 3C is a diagram for explaining an amount of displacement Q1 and an amount of displacement Q2 in a first image;

FIG. 3D is a diagram for explaining a first amount of rotation and a second amount of rotation;

FIG. 4A is a schematic perspective view of a first example of the motor-driven device, which supports an imaging device;

FIG. 4B is a schematic perspective view of a second example of the motor-driven device, which supports the imaging device;

FIG. 4C is a schematic perspective view of a third example of the motor-driven device, which supports the imaging device;

FIG. 5 is a diagram schematically illustrating examples of imaging of a living body by an imaging system according to a modification of the present embodiment;

FIG. 6A is a diagram illustrating a comparative example in which cerebral blood flow information of a target portion after movement was obtained in a state in which the orientation of the imaging device was fixed;

FIG. 6B is a diagram illustrating a working example in which cerebral blood flow information of a target portion after movement was obtained after the orientation of the imaging device was changed in accordance with movement of a living body;

FIG. 7 is a diagram illustrating an example configuration of the second imaging device;

FIG. 8A is a diagram illustrating an example operation of emitting a first light pulse and a second light pulse;

FIG. 8B is a diagram illustrating another example operation of emitting the first light pulse and the second light pulse;

FIG. 9A is a diagram schematically illustrating example changes over time of a surface-reflected component and an internal scattered component included in a reflected-light pulse when a light pulse has an impulse waveform;

FIG. 9B is a diagram schematically illustrating example changes over time of the surface-reflected component and the internal scattered component included in the reflected-light pulse when the light pulse has a rectangular waveform; and

FIG. 9C is a flowchart schematically illustrating operations of the processing device performed in relation to the first light source, the second light source, and the second imaging device.

### DETAILED DESCRIPTIONS

Any of the embodiments described below is a general or specific example. Numerical values, shapes, materials, constituent elements, the positions and connections of constituent elements, steps, and the order of steps described in the following embodiments are illustrative and are not intended to limit the techniques of the present disclosure. Among the constituent elements described in the following embodiments, a constituent element not described in an independent claim stating the most generic concept will be described as an optional constituent element. Each of the diagrams is a schematic diagram and is not necessarily a precise diagram. Substantially the same or similar constituent elements in the diagrams are assigned the same reference numerals. A duplicated description may be omitted or briefly given.

First, an overview of embodiments of the present disclosure will be briefly described.

There may be a demand for obtaining, in an environment in which a living body moves, biological information of a target portion of the living body, as in an example case where a person who is working or driving a vehicle is assumed to be a target object and surface blood flow information of the forehead and/or cerebral blood flow information is obtained. When the orientation of an imaging device that obtains biological information is fixed, biological information of the target portion after movement might not be stably obtained.

An imaging system according to an embodiment of the present disclosure includes a first imaging device having a relatively wide field of view for obtaining positional information of a living body and a second imaging device having a relatively narrow field of view for obtaining biological information of a target portion of a living body. With this imaging system, the orientation of the second imaging device can be changed so as to allow imaging of a target portion of a living body after movement, on the basis of positional information of the living body obtained by the first imaging device. As a result, in an environment in which a living body moves, biological information of a target portion of the living body can be stably obtained in a noncontact manner. An imaging system, a processing device, and a method to be performed by a computer in an imaging system according to embodiments of the present disclosure will be described below.

An imaging system according to a first item includes: a first imaging device that has a first field of view; a second imaging device that has a second field of view narrower than the first field of view; and a motor-driven device that is capable of changing an orientation of the second imaging device. The first imaging device images a living body and generates first image data. The second imaging device images a target portion of the living body and generates second image data. The second image data is sent to a processing device that generates, on the basis of the second image data, data indicating biological information of the target portion. The motor-driven device changes the orientation of the second imaging device on the basis of a position of the living body in an image based on the first image data and maintains a state in which the target portion is included in the second field of view.

With this imaging system, in an environment in which a living body moves, biological information of a target portion of the living body can be stably obtained in a noncontact manner.

An imaging system according to a second item is the imaging system according to the first item, in which the motor-driven device is capable of changing an orientation of the first imaging device. The motor-driven device changes the orientation of the first imaging device and the orientation of the second imaging device synchronously on the basis of the position of the living body in the image based on the first image data.

With this imaging system, the relative positional relationship between the second field of view and the target portion can be known on the basis of the first image data indicating the position of the living body in the first field of view, regardless of the orientations of the first imaging device and the second imaging device.

An imaging system according to a third item is the imaging system according to the second item, in which the imaging system includes the processing device.

With this imaging system, data indicating biological information of the target portion can be generated by the processing device.

An imaging system according to a fourth item is the imaging system according to the third item, in which the image based on the first image data includes a face of the living body. The processing device causes the motor-driven device to change the orientation of the first imaging device such that a specific position of the image based on the first image data is included in a region of the face of the living body.

With this imaging system, the orientations of the first imaging device and the second imaging device change synchronously, and the target portion can consequently fit inside the second field of view.

An imaging system according to a fifth item is the imaging system according to the fourth item, in which the processing device causes the motor-driven device to change the orientation of the first imaging device and subsequently causes the motor-driven device to further change the orientation of the first imaging device so as to decrease an amount of displacement between the specific position of the image based on the first image data and a specific position of the face of the living body.

With this imaging system, the amount of displacement can be further reduced.

An imaging system according to a sixth item is the imaging system according to any of the second to fifth items, in which the target portion includes a forehead of the living body. The processing device causes the motor-driven device to change the orientation of the second imaging device such that the second field of view includes the forehead and eyebrows of the living body.

With this imaging system, in a correction for making the position of the target portion before movement of the living body and the position of the target portion after movement of the living body coincide with each other by image-processing-based tracking, the edge portions of the eyebrows can be used as feature points.

An imaging system according to a seventh item is the imaging system according to any of the second to sixth items, in which the processing device causes the motor-driven device to change the orientation of the second imaging device such that the second field of view includes the target portion, and subsequently determines a pixel region of a portion corresponding to the target portion in an image based on the second image data.

With this imaging system, biological information of the target portion can be obtained from the determined pixel region.

An imaging system according to an eighth item is the imaging system according to the seventh item, in which the pixel region coincides with a pixel region of a portion corresponding to the target portion in an image based on the second image data before movement of the living body.

With this imaging system, even when the living body moves, biological information of the same target portion before movement of the living body can be obtained.

An imaging system according to a ninth item is the imaging system according to any of the first to eighth items, in which the biological information is cerebral blood flow information of the living body.

With this imaging system, cerebral blood flow information of the living body can be obtained.

An imaging system according to a tenth item is the imaging system according to any of the first to ninth items, including at least one light source that emits a light pulse for irradiating the target portion of the living body.

With this imaging system, biological information of the target portion can be obtained by irradiating the target portion of the living body.

A processing device according to an eleventh item is a processing device to be used in an imaging system. The imaging system includes: a first imaging device that has a first field of view, a second imaging device that has a second field of view narrower than the first field of view, and a motor-driven device that is capable of changing an orientation of the second imaging device. The processing device includes: a processor; and a memory that stores a computer program to be executed by the processor. The computer program causes the processor to cause the first imaging device to image a living body and to generate first image data, cause the motor-driven device to change the orientation of the second imaging device on the basis of a position of the living body in an image based on the first image data and to maintain a state in which a target portion of the living body is included in the second field of view, cause the second imaging device to image the target portion and to generate second image data, and generate data indicating biological information of the target portion on the basis of the second image data.

With this processing device, in an environment in which a living body moves, biological information of a target portion of the living body can be stably obtained in a noncontact manner.

A processing device according to a twelfth item is the processing device according to the eleventh item, in which the motor-driven device is capable of changing an orientation of the first imaging device. Changing the orientation of the second imaging device on the basis of the position of the living body in the image based on the first image data includes changing the orientation of the first imaging device and the orientation of the second imaging device synchronously on the basis of the position of the living body in the image based on the first image data.

With this processing device, the relative positional relationship between the second field of view and the target portion can be known on the basis of the first image data indicating the position of the living body in the first field of view, regardless of the orientations of the first imaging device and the second imaging device.

A method according to a thirteenth item is a method to be performed by a computer in an imaging system. The imaging system includes: a first imaging device that has a first field of view, a second imaging device that has a second field of view narrower than the first field of view, and a motor-driven device that is capable of changing an orientation of the second imaging device. The method includes: causing the first imaging device to image a living body and to generate first image data; causing the motor-driven device to change the orientation of the second imaging device on the basis of a position of the living body in an image based on the first image data and to maintain a state in which a target portion of the living body is included in the second field of view; causing the second imaging device to image the target portion and to generate second image data; and generating data indicating biological information of the target portion on the basis of the second image data.

With this method, in an environment in which a living body moves, biological information of a target portion of the living body can be stably obtained in a noncontact manner.

A method according to a fourteenth item is the method according to the thirteenth item, in which the motor-driven device is capable of changing an orientation of the first imaging device. Changing the orientation of the second imaging device on the basis of the position of the living body in the image based on the first image data includes changing the orientation of the first imaging device and the orientation of the second imaging device synchronously on the basis of the position of the living body in the image based on the first image data.

With this method, the relative positional relationship between the second field of view and the target portion can be known on the basis of the first image data indicating the position of the living body in the first field of view, regardless of the orientations of the first imaging device and the second imaging device.

In the present disclosure, all or some of the circuits, units, apparatuses, members, or sections or all or some of the functional blocks in block diagrams can be implemented as, for example, one or more electronic circuits that include a semiconductor device, a semiconductor integrated circuit (IC), or an LSI (Large Scale Integration) circuit. An LSI circuit or an IC may be integrated into a single chip or may be constituted by a combination of chips. For example, functional blocks other than a memory cell may be integrated into a single chip. Although the circuit is called an LSI circuit or an IC here, the circuit is called differently depending on the degree of integration, and the circuit may be one that is called a system LSI circuit, a VLSI (Very Large Scale Integration) circuit, or a ULSI (Ultra Large Scale Integration) circuit. A field-programmable gate array (FPGA) that can be programmed after LSI manufacturing or a reconfigurable logic device that allows reconfiguration of the connections inside the LSI circuit or setup of circuit cells inside the LSI circuit can be used for the same purpose.

Furthermore, all or some of the functions or operations of any circuit, unit, apparatus, member, or section can be implemented as software processing. In this case, software is recorded in one or more ROMs, optical discs, hard disk drives, or other non-transitory storage media, and when the software is executed by a processor, functions implemented as the software are executed by the processor and a peripheral device. A system or an apparatus may include one or more non-transitory storage media in which the software is recorded, the processor, and a necessary hardware device, such as an interface.

In the present disclosure, “light” means not only visible light (having a wavelength of about 400 nm to about 700 nm) but also electromagnetic waves including ultraviolet rays (having a wavelength of about 10 nm to about 400 nm) and infrared rays (having a wavelength of about 700 nm to about 1 mm).

Hereinafter, more specific embodiments of the present disclosure will be described with reference to the drawings.

### EMBODIMENTS

### Imaging System

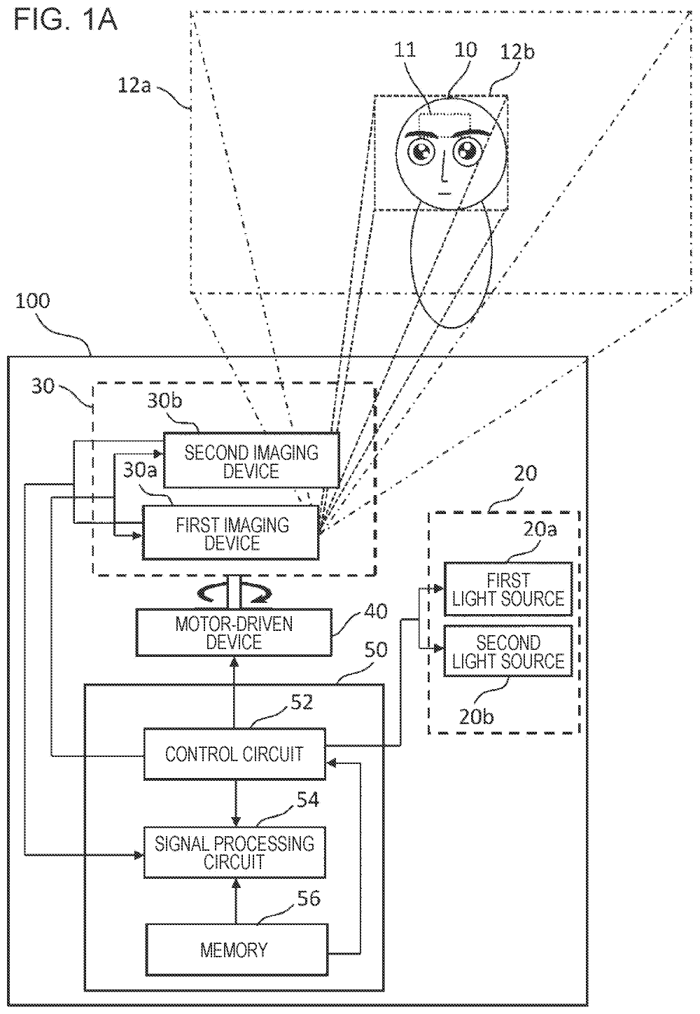

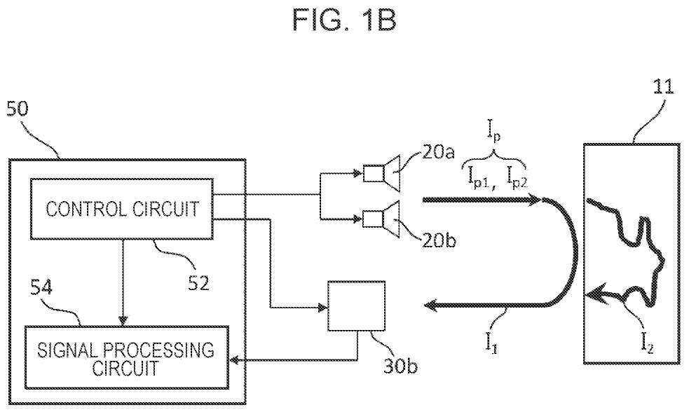

A configuration of an imaging system according to an embodiment of the present disclosure will first be described with reference to FIG. 1A and FIG. 1B. FIG. 1A is a block diagram schematically illustrating a configuration of an imaging system according to an exemplary embodiment of the present disclosure. FIG. 1A illustrates a person as a living body 10 and illustrates a human head and a human trunk. The living body 10 is illuminated with ambient light, such as illuminating light or sunlight. The living body 10 does not always stand still and may move as in a case of, for example, working or driving a vehicle. The living body 10 is not limited to a person and may be, for example, an animal. The region outlined by a dotted line in FIG. 1A represents a target portion 11 of the living body 10.

An imaging system 100 illustrated in FIG. 1A includes a first light source 20a, a second light source 20b, a first imaging device 30a, a second imaging device 30b, a motor-driven device 40, and a processing device 50. The processing device 50 includes a control circuit 52, a signal processing circuit 54, and a memory 56. In the specification, the first light source 20a and the second light source 20b are also referred to as “light source 20” without distinguishing the light sources from each other. Similarly, the first imaging device 30a and the second imaging device 30b are also referred to as “imaging device 30” without distinguishing the imaging devices from each other. FIG. 1B is a diagram schematically illustrating the first light source 20a, the second light source 20b, the second imaging device 30b, and the control circuit 52 and the signal processing circuit 54 included in the processing device 50, which are included in the imaging system 100 in FIG. 1A. FIG. 1B includes an enlarged view of the target portion 11 of the living body 10.

The light source 20 emits a light pulse for irradiating the target portion 11 of the living body 10. The first imaging device 30a has a relatively wide first field of view 12a and obtains positional information of the living body 10 from reflected light produced as a result of the above-described ambient light being reflected at the living body 10. The second imaging device 30b has a relatively narrow second field of view 12b and obtains biological information of the target portion 11 from a reflected-light pulse produced as a result of the light pulse being reflected at the target portion 11 of the living body 10. The second field of view 12b is positioned inside the first field of view 12a. In FIG. 1A, the region outlined by a one-dot chain line represents the first field of view 12a, and the region outlined by a dashed line represents the second field of view 12b. The motor-driven device 40 supports the first imaging device 30a and the second imaging device 30b and changes the orientation of the imaging device 30 in response to a signal, from the processing device 50, based on the positional information of the living body 10. The motor-driven device 40 changes the orientation of the imaging device 30 on the basis of the signal to thereby maintain a state in which the living body 10 is included in the first field of view 12a and the target portion 11 of the living body 10 is included in the second field of view 12b even after movement of the living body 10. As a result, biological information of the target portion 11 of the living body 10 can be stably obtained in a noncontact manner. The biological information can be, for example, cerebral blood flow information or face or scalp blood flow information of the living body 10.

The constituent elements of the imaging system 100 in the present embodiment will be described in detail below.

### First Light Source 20a and Second Light Source 20b

The first light source 20a emits a first light pulse Iₚ₁ for irradiating the target portion 11 as illustrated in FIG. 1B. The first light pulse Iₚ₁ has a first wavelength. Similarly, the second light source 20b emits a second light pulse Iₚ₂ for irradiating the target portion 11 as illustrated in FIG. 1B. The second light pulse Iₚ₂ has a second wavelength longer than the first wavelength. In the example illustrated in FIG. 1A and FIG. 1B, the number of first light sources 20a is one but may be more than one. In the example illustrated in FIG. 1A and FIG. 1B, the number of second light sources 20b is one but may be more than one. Depending on the use, both of the first light source 20a and the second light source 20b need not be used and one of the light sources may be used.

In the specification, the first light pulse Iₚ₁ and the second light pulse Iₚ₂ are also referred to as “light pulse Iₚ” without distinguishing the light pulses from each other. The light pulse Iₚ includes a rising portion and a falling portion. The rising portion is a portion, of the light pulse Iₚ, from the start of an increase in the intensity to the end of the increase. The falling portion is a portion, of the light pulse Iₚ, from the start of a decrease in the intensity to the end of the decrease.

A portion of the light pulse Iₚ reaching the target portion 11 becomes a surface-reflected component I₁, which is reflected on the surface of the target portion 11, and another portion thereof becomes an internal scattered component I₂, which is reflected or scattered one time or multiply scattered inside the target portion 11. The surface-reflected component I₁ includes three components, namely, a directly reflected component, a diffused-reflected component, and a scattered-reflected component. The directly reflected component is a reflected component having an angle of incidence and an angle of reflection that are equal to each other. The diffused-reflected component is a component that is diffused and reflected due to irregularities of the surface. The scattered-reflected component is a component that is scattered and reflected by internal tissue in the vicinity of the surface. When the target portion 11 is the forehead of the living body 10, the scattered-reflected component is a component that is scattered and reflected inside the epidermis. A description will be given below under the assumption that the surface-reflected component I₁ reflected on the surface of the target portion 11 includes these three components. A description will be given under the assumption that the internal scattered component I₂ does not include a component that is scattered and reflected by internal tissue in the vicinity of the surface. The surface-reflected component I₁ and the internal scattered component I₂ are reflected or scattered, the directions of travel of these components change, and a portion of the surface-reflected component I₁ and a portion of the internal scattered component I₂ reach the second imaging device 30b as a reflected-light pulse. The surface-reflected component I₁ reflects surface information of the living body 10, such as face and scalp blood flow information. From the face and scalp blood flow information, for example, the external appearance of the face, the skin blood flow, the heart rate, or the sweat rate of the living body 10 can be known. The internal scattered component I₂ reflects internal information of the living body 10, such as cerebral blood flow information. From the cerebral blood flow information, for example, the cerebral blood flow, the blood pressure, the blood oxygen saturation level, or the heart rate of the living body 10 can be known. “Detecting the surface-reflected component I₁” may be construed as “detecting a portion of the surface-reflected component I₁”. “Detecting the internal scattered component I₂” may be construed as “detecting a portion of the internal scattered component I₂”. A method for detecting the internal scattered component I₂ from the reflected-light pulse will be described below.

Each of the first wavelength of the first light pulse Iₚ₁ and the second wavelength of the second light pulse Iₚ₂ can be any wavelength included in a wavelength range of, for example, greater than or equal to 650 nm and less than or equal to 950 nm. This wavelength range is included in the wavelength range of red to near-infrared rays. The above-described wavelength range is called “biological window” and has a property that light in the wavelength range is relatively less likely to be absorbed into moisture inside the living body and into the skin. When a living body is a detection target, the sensitivity of detection can be increased by using light in the above-described wavelength range. When a change in the cerebral blood flow of a user is detected, light that is used is considered to be absorbed mainly into oxygenated hemoglobin (HbO₂) and deoxygenated hemoglobin (Hb). In general, when a blood flow changes, the concentration of oxygenated hemoglobin and the concentration of deoxygenated hemoglobin change. With these changes, the degree of absorption of light also changes. Therefore, when a blood flow changes, the amount of light detected changes over time.

Oxygenated hemoglobin and deoxygenated hemoglobin differ in the wavelength dependence of light absorption. For a wavelength of greater than or equal to 650 nm and shorter than 805 nm, the coefficient of light absorption by deoxygenated hemoglobin is larger than the coefficient of light absorption by oxygenated hemoglobin. For a wavelength of 805 nm, the coefficient of light absorption by deoxygenated hemoglobin and the coefficient of light absorption by oxygenated hemoglobin are equal to each other. For a wavelength of longer than 805 nm and less than or equal to 950 nm, the coefficient of light absorption by oxygenated hemoglobin is larger than the coefficient of light absorption by deoxygenated hemoglobin.

Therefore, when the first wavelength of the first light pulse Iₚ₁ is set to a wavelength of greater than or equal to 650 nm and shorter than 805 nm, the second wavelength of the second light pulse Iₚ₂ is set to a wavelength of longer than 805 nm and less than or equal to 950 nm, and the target portion 11 is irradiated with the first light pulse Iₚ₁ and the second light pulse Iₚ₂, the concentration of oxygenated hemoglobin and the concentration of deoxygenated hemoglobin contained in blood inside the target portion 11 can be obtained by processing by the processing device 50 described below. With irradiation with two light pulses having different wavelengths, more detailed internal information of the target portion 11 can be obtained.

In the present embodiment, the light source 20 can be designed by taking into consideration an influence on the user's retina. For example, the light source 20 can be a laser source, such as a laser diode, and can satisfy Class 1 of a laser safety standard developed by each country. When Class 1 is satisfied, the target portion 11 is irradiated with low-illuminance light having an accessible emission limit (AES) of less than 1 mW. Note that the light source 20 itself need not satisfy Class 1. Class 1 of the laser safety standard may be satisfied by, for example, placing a diffuser plate or an ND filter in front of the light source 20 and diffusing or attenuating light.

### First Imaging Device 30a and Second Imaging Device 30b

The first imaging device 30a obtains positional information of the living body 10 from reflected light produced as a result of ambient light being reflected at the living body 10. The first imaging device 30a images the living body 10, generates first image data, and sends the first image data to the processing device 50. The first image data need not be data of an image and may be raw data of the pixel values of pixels distributed in two dimensions. The pixels correspond to the pixel values on a one-to-one basis. The first image data reflects positional information of the living body 10. An image based on the first image data is referred to as “first image”. Even when the target portion 11 moves outside the second field of view 12b, as long as the living body 10 is present within the first field of view 12a, the first imaging device 30a can follow the living body 10. The first imaging device 30a can be, for example, a monochrome camera or an RGB camera.

The second imaging device 30b obtains biological information of the target portion 11 of the living body 10 from a reflected-light pulse produced as a result of the light pulse Iₚ being reflected at the target portion 11 of the living body 10. The second imaging device 30b images the target portion 11 of the living body 10, generates second image data, and sends the second image data to the processing device 50. The second image data need not be data of an image and may be raw data of the pixel values of pixels distributed in two dimensions similarly to the first image data. The pixels correspond to the pixel values on a one-to-one basis. The second image data reflects biological information of the target portion 11 of the living body 10. An image based on the second image data is referred to as “second image”. When the second field of view 12b is made narrower than the first field of view 12a, the number of pixels of the target portion 11 included in the second image can be made larger than the number of pixels of the target portion 11 included in the first image. Therefore, when the pixel values of the pixels in the second image are subjected to addition averaging, noise can be reduced and the SN ratio of imaging can be improved.

The second imaging device 30b can include pixels arranged in two dimensions on an imaging surface. Each pixel can include a photoelectric transducer, such as a photodiode, and one or more charge storage units. The second imaging device 30b can be any image sensor, such as a CCD image sensor or a CMOS image sensor. The configuration of the second imaging device 30b will be described in detail below.

The second imaging device 30b detects at least a component of a reflected-light pulse, in a rising period, produced as a result of the light pulse Iₚ being reflected at the target portion 11 and outputs a signal corresponding to the intensity of the component. The signal reflects surface information of the target portion 11. Alternatively, the second imaging device 30b detects at least a component of a reflected-light pulse, in a falling period, produced as a result of the light pulse Iₚ being reflected at the target portion 11 and outputs a signal corresponding to the intensity of the component. The signal reflects internal information of the target portion 11.

The “rising period” of a reflected-light pulse refers to a period from the point of time when the intensity of the reflected-light pulse starts increasing to the point of time when the intensity stops increasing on the imaging surface of the second imaging device 30b. The “falling period” of a reflected-light pulse refers to a period from the point of time when the intensity of the reflected-light pulse starts decreasing to the point of time when the intensity stops decreasing on the imaging surface of the second imaging device 30b. More precisely, the “rising period” means a period from the point of time when the intensity of the reflected-light pulse exceeds a preset lower limit to the point of time when the intensity reaches a preset upper limit. The “falling period” means a period from the point of time when the intensity of the reflected-light pulse falls below the preset upper limit to the point of time when the intensity reaches the preset lower limit. The upper limit can be set to a value equal to, for example, 90% of the peak value of the intensity of the reflected-light pulse, and the lower limit can be set to a value equal to, for example, 10% of the peak value.

The second imaging device 30b can include an electronic shutter. The electronic shutter is a circuit that controls the timing of imaging. The electronic shutter controls one signal storage period in which received light is converted to an effective electric signal and the signal is stored and a period in which signal storage is stopped. The signal storage period is also referred to as “exposure period”. In the following description, the width of the exposure period is also referred to as “shutter width”. The time from when one exposure period ends to when the next exposure period starts is also referred to as “non-exposure period”.

The second imaging device 30b can adjust the exposure period and the non-exposure period with the electronic shutter in units of sub-nanoseconds in a range of, for example, 30 ps to 1 ns. An existing TOF (Time-of-Flight) camera for distance measurement detects all light rays emitted from the light source 20, reflected at a subject, and returning thereto. An existing TOF camera needs to have a shutter width larger than the pulse width of light. In contrast, the imaging system 100 in the present embodiment need not correct the amount of light of a subject. Therefore, the shutter width need not be larger than the pulse width of the reflected-light pulse. The shutter width can be set to a value of, for example, greater than or equal to 1 ns and less than or equal to 30 ns. With the imaging system 100 in the present embodiment, the shutter width can be reduced and an influence of a dark current included in a detection signal can be reduced.

### Motor-Driven Device 40

The motor-driven device 40 supports the imaging device 30 and can change the orientation of the imaging device 30 by panning rotation and/or tilting rotation by a motor. Panning rotation can move the field of view of the imaging device 30 in the horizontal direction, and tilting rotation can move the field of view of the imaging device 30 in the vertical direction. An operation of changing the orientation of the imaging device 30 by panning rotation is called “panning correction”, and an operation of changing the orientation of the imaging device 30 by tilting rotation is called “tilting correction”.

The motor-driven device 40 changes, in response to a signal from the processing device 50, the orientation of the imaging device 30 so as to follow movement of the living body 10 in the first image. With this operation by the motor-driven device 40, even after up-and-down and side-to-side movement of the living body 10, a state in which the living body 10 is included in the first field of view 12a and the target portion 11 of the living body 10 is included in the second field of view 12b can be maintained. The motor-driven device 40 can, for example, change the orientation of the first imaging device 30a and the orientation of the second imaging device 30b synchronously. In this case, the relative positional relationship between the first field of view 12a and the second field of view 12b does not depend on the orientation of the imaging device 30. Therefore, based on the first image data indicating the position of the living body 10 in the first field of view 12a, the relative positional relationship between the second field of view 12b and the target portion 11 can be known. Depending on the use, the motor-driven device 40 may change the orientation of the second imaging device 30b without changing the orientation of the first imaging device 30a.

The motor-driven device 40 can include, for example, at least one motor selected from the group consisting of a DC motor, a brushless DC motor, a PM motor, a stepping motor, an induction motor, a servo motor, an ultrasonic motor, an AC motor, and an in-wheel motor. The motor-driven device 40 may include a motor for panning rotation and a motor for tilting rotation separately. The motor-driven device 40 may rotate the imaging device 30 in a rolling direction with a motor. The rolling direction means a direction about a rotation axis perpendicular to the rotation axis of panning rotation and the rotation axis of tilting rotation. When the living body 10 inclines the face, a state in which the target portion 11 of the living body 10 is included in the second field of view 12b can be maintained by rotating the imaging device 30 in the rolling direction so as to follow the inclination of the face on the basis of the first image data. The detailed configuration of the motor-driven device 40 will be described below.

### Processing Device 50

The control circuit 52 included in the processing device 50 controls operations of the light source 20, the imaging device 30, and the signal processing circuit 54. The control circuit 52 adjusts the time difference between the emission timing of the light pulse Iₚ of the light source 20 and the shutter timing of the second imaging device 30b. In the specification, the time difference is also referred to as “phase difference”. The “emission timing” of the light source 20 is the timing when the light pulse emitted from the light source 20 starts rising. The “shutter timing” is the timing when exposure starts. The control circuit 52 may adjust the phase difference by changing the emission timing or may adjust the phase difference by changing the shutter timing.

The control circuit 52 may be configured to remove an offset component from a signal detected by each pixel of the second imaging device 30b. The offset component is a signal component resulting from ambient light, such as sunlight or illuminating light, or disturbance light. When a signal is detected by the second imaging device 30b in a state in which driving of the light source 20 is turned OFF and light is not emitted from the light source 20, an offset component resulting from ambient light or disturbance light can be estimated.

The signal processing circuit 54 included in the processing device 50 generates and outputs data indicating positional information of the living body 10, on the basis of the first image data. From the data, the positions of the living body 10 and the target portion 11 in the first image can be identified. The signal processing circuit 54 generates and outputs data indicating biological information of the target portion 11 of the living body 10, on the basis of the second image data. The data reflects surface information and/or internal information of the target portion 11. A method for calculating, as the internal information, the amount of change in the concentration of each of HbO₂ and Hb in the cerebral blood from an initial value will be described in detail below.

The signal processing circuit 54 can estimate the mental condition and/or the physical condition of the living body 10 on the basis of the surface information and/or the internal information of the target portion 11. The signal processing circuit 54 may generate and output data indicating the mental condition and/or the physical condition of the living body 10. The mental condition can be, for example, a mood, a feeling, a state of health, or a temperature sense. The mood can include, for example, a pleasant mood or an unpleasant mood. The feeling can include, for example, a feeling of security, a feeling of anxiety, sadness, or anger. The state of health can include, for example, a vital state or a fatigue state. The temperature sense can include, for example a sense of hotness, a sense of coldness, or a sense of humid heat. As derivatives of these, indicators indicating the degree of cerebral activity, such as the degree of interest, the degree of proficiency, the degree of mastery, and the degree of concentration, can also be included in the mental condition. The physical condition can be, for example, the degree of fatigue, sleepiness, or the degree of being drunk.

The control circuit 52 can be, for example, a combination of a processor and a memory or an integrated circuit, such as a micro-controller, including a processor and a memory. For example, the control circuit 52 adjusts the emission timing and the shutter timing and causes the signal processing circuit 54 to perform signal processing by, for example, the processor executing a computer program recorded in the memory 56.

The signal processing circuit 54 can be implemented as a digital signal processor (DSP), a programmable logic device (PLD), such as a field-programmable gate array (FPGA), or a combination of a central processing unit (CPU) or a graphics processing unit (GPU) and a computer program. The signal processing circuit 54 performs signal processing by the processor executing a computer program recorded in the memory 56.

The signal processing circuit 54 and the control circuit 52 may be implemented as one integrated circuit or as separate individual circuits. At least one of the signal processing circuit 54, the control circuit 52, and the memory 56 may be implemented as a constituent element of a remotely installed external apparatus, such as a server. In this case, the external apparatus, such as a server, mutually transmits and receives data to and from the other constituent elements by wireless communication or wired communication.

In the specification, operations of the control circuit 52 and the signal processing circuit 54 will be described together as operations of the processing device 50.

### Others

The imaging system 100 may include a first image forming optical system that forms a two-dimensional image of the living body 10 on an imaging surface of the first imaging device 30a and a second image forming optical system that forms a two-dimensional image of the target portion 11 on the imaging surface of the second imaging device 30b. The optical axis of the first image forming optical system is substantially orthogonal to the imaging surface of the first imaging device 30a. The optical axis of the second image forming optical system is substantially orthogonal to the imaging surface of the second imaging device 30b. Each of the first and second image forming optical systems may include a zoom lens. When the focal length is changed by using the zoom lens of the first image forming optical system, the resolution of a two-dimensional image of the living body 10 imaged by the first imaging device 30a changes. When the focal length is changed by using the zoom lens of the second image forming optical system, the resolution of a two-dimensional image of the living body 10 imaged by the second imaging device 30b changes. Therefore, even when the distance to the living body 10 is long, a desired measurement region can be enlarged and observed in detail.

The imaging system 100 may include, between the target portion 11 and the second imaging device 30b, a bandpass filter that allows light in a wavelength band emitted from the light source 20, or light in a wavelength band emitted from the light source 20 and light in the vicinity of the wavelength band to pass therethrough. This can reduce an influence of a disturbance component, such as ambient light. The bandpass filter can be constituted by, for example, a multi-layer filter or an absorption filter. Taking into consideration a band shift associated with a temperature change in the light source 20 and oblique incidence on the filter, the bandwidth of the bandpass filter may be in a range of greater than or equal to 20 nm and less than or equal to 100 nm approximately.

In a case of obtaining internal information, the imaging system 100 may include a first polarizing plate between the target portion 11 and the light source 20 and a second polarizing plate between the target portion 11 and the second imaging device 30b. In this case, the polarization direction of the first polarizing plate and the polarization direction of the second polarizing plate can have a crossed Nicols relationship. The disposition of these two polarizing plates can prevent a regular reflection component in the surface-reflected component I₁ from the target portion 11, that is, a component having an angle of incidence and an angle of reflection that are equal to each other, from reaching the second imaging device 30b. That is, the amount of light of the surface-reflected component I₁ reaching the second imaging device 30b can be reduced.

### Correction Operation Performed by Processing Device 50

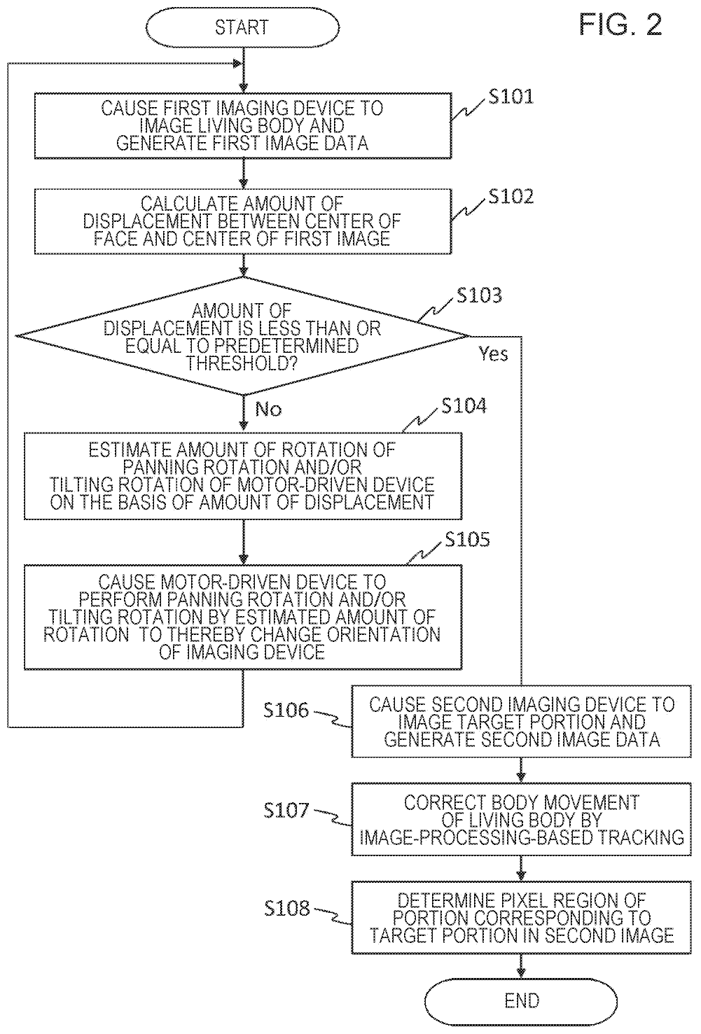

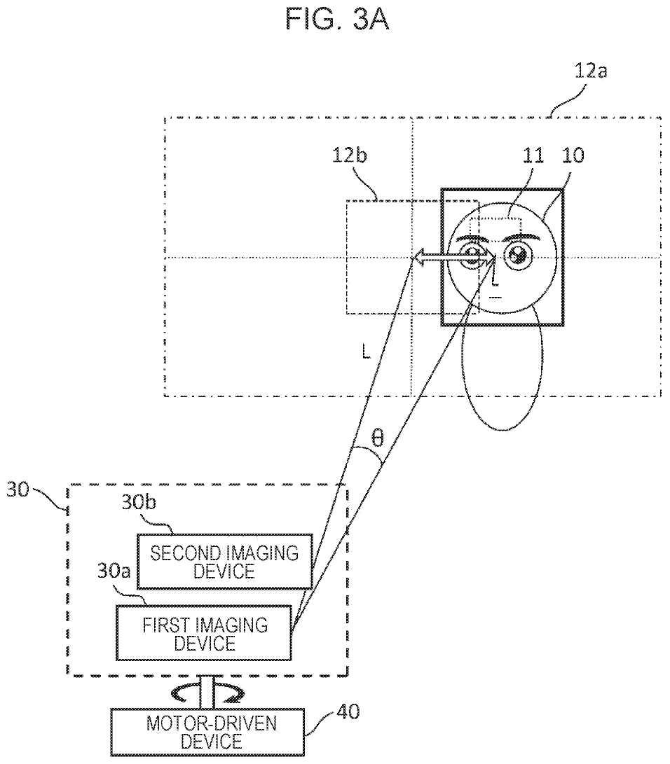

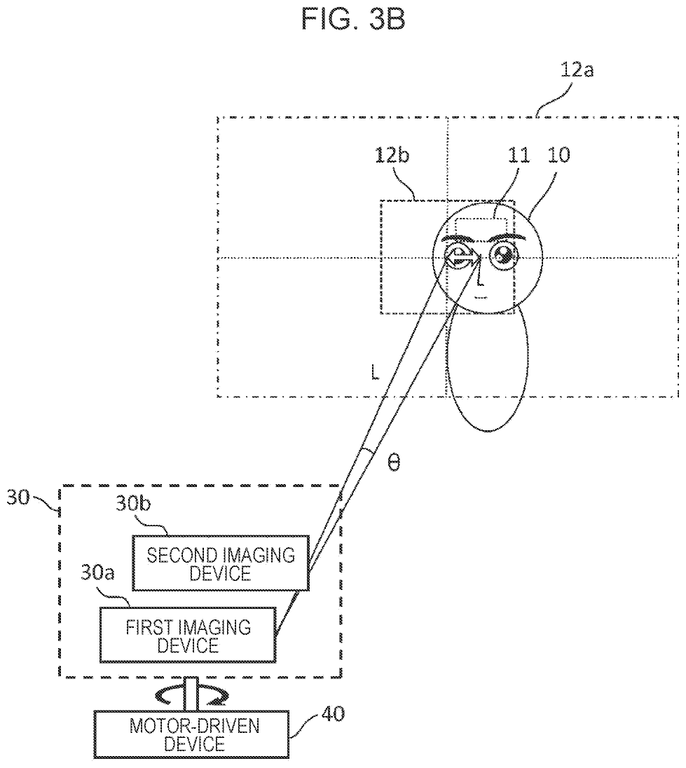

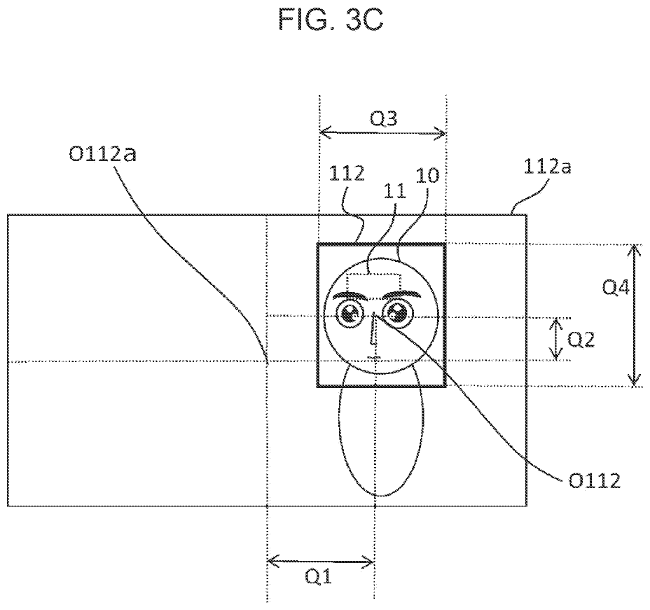

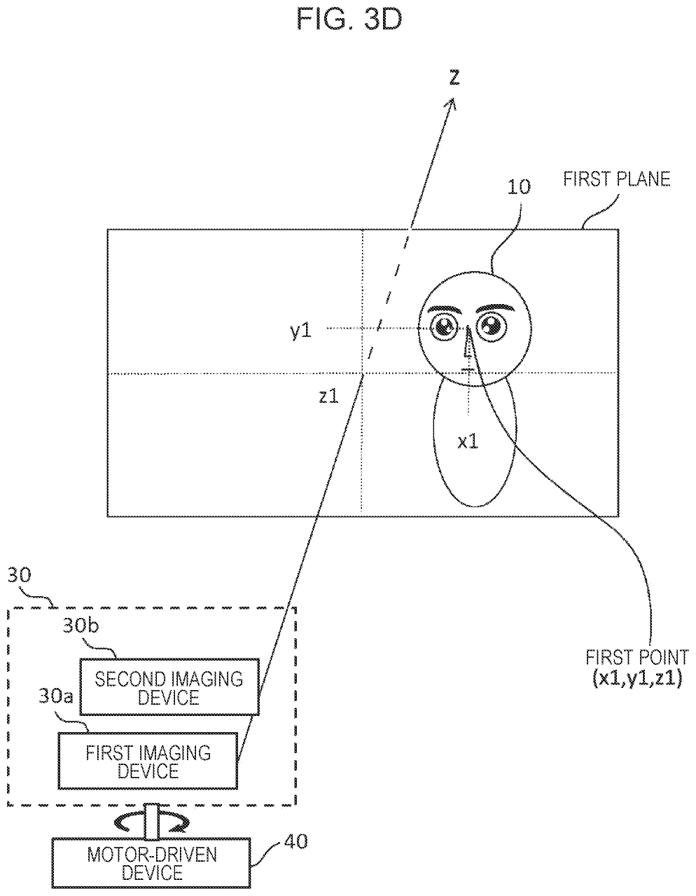

An example correction operation performed by the processing device 50 when the living body 10 moves will now be described with reference to FIG. 2 to FIG. 3B. FIG. 2 is a flowchart schematically illustrating an example correction operation performed by the processing device 50 when the living body 10 moves. The processing device 50 performs operations in step S101 to S108 illustrated in FIG. 2. FIG. 3A and FIG. 3B are diagrams for explaining operations of the motor-driven device 40.

### Step S101

In step S101, the processing device 50 causes the first imaging device 30a to image the living body 10 and generate and output first image data. In the first image, an object present inside the first field of view 12a is present. The first image includes the face of the living body 10.

### Step S102

In step S102, the processing device 50 extracts the face of the living body 10 from the first image by a machine learning process on the basis of the first image data and calculates the amount of displacement between the center of the extracted face and the center of the first image.

The processing device 50 has a cascade classifier that has learned human faces. The classifier reads the first image data, encloses the face portion of the living body 10 in the first image within a rectangular frame, and outputs the coordinates of the frame in the first image. The thick rectangular frame illustrated in FIG. 3A corresponds to the rectangular frame in the first image. The hollow double-sided arrow illustrated in FIG. 3A represents the amount of displacement between the center of the face in the first field of view 12a and the center of the first field of view 12a and corresponds to the amount of displacement between the center of the face in the first image and the center of the first image.

### Step S103

In step S103, the processing device 50 determines whether the amount of displacement between the center of the face in the first image and the center of the first image is less than or equal to a predetermined threshold. The predetermined threshold can be, for example, less than or equal to ½ of the width of the face extracted by the machine learning process. When the amount of displacement is less than or equal to ½ of the width of the face, the center of the first image can be included in the region of the extracted face, and the face can be disposed in a substantially central portion of the first image. If determination in step S103 results in No, the processing device 50 performs the operation in step S104. If determination in step S103 results in Yes, the processing device 50 performs the operation in step S106.

### Step S104

In step S104, the processing device 50 estimates the amount of rotation of panning rotation and/or tilting rotation of the motor-driven device 40 on the basis of the amount of displacement. As illustrated in FIG. 3A, based on a distance L and the amount of displacement between the center of the face in the first field of view 12a and the center of the first field of view 12a, an angle of rotation θ by which a correction is to be made to some extent can be calculated. The distance L is the distance between the center of the imaging surface of the first imaging device 30a and the center of the first field of view 12a. The amount of displacement between the center of the face in the first field of view 12a and the center of the first field of view 12a can be known by associating the number of pixels of the amount of displacement between the center of the face in the first image and the center of the first image and the actual distance.

In general, when an optical lens has no distortion, a real image height h on an imaging element surface is expressed by h=f×tan(θ), where θ is the angle of view on the object side and f is the focal length of the optical lens. This is taken into consideration, and the processing device 50 may estimate the amount of rotation of panning rotation and/or tilting rotation of the motor-driven device 40 as follows in step S104.

The processing device 50 calculates the angle of rotation θ by which a correction is to be made, on the basis of the focal length f of an optical lens built in the first imaging device 30a and the amount of displacement h between the center of the face in the first field of view 12a and the center of the first field of view 12a, formed on the first imaging device 30a. The amount of displacement h between the center of the face in the first field of view 12a and the center of the first field of view 12a formed on the first imaging device 30a can be known by associating the number of pixels of the amount of displacement between the center of the face in the first image and the center of the first image and the pixel size. The angle of rotation θ by which a correction is to be made can be calculated as θ=a tan(h/f)

### Step S105

In step S105, the processing device 50 causes the motor-driven device 40 to perform panning rotation and/or tilting rotation by the estimated amount of rotation as illustrated in FIG. 3B to thereby change the orientation of the first imaging device 30a and the orientation of the second imaging device 30b synchronously.

The processing device 50 repeats the operations in steps S101 to S105 until the amount of displacement becomes less than or equal to the threshold. In other words, the processing device 50 causes the motor-driven device 40 to change the orientation of the first imaging device 30a and the orientation of the second imaging device 30b synchronously, and subsequently repeats the operations of causing the motor-driven device 40 to further change the orientations of the imaging devices synchronously so as to decrease the amount of displacement. As described above, when the threshold is less than or equal to ½ of the width of the face, the center of the first image can be included in the region of the extracted face. Therefore, in other words, the processing device 50 causes the motor-driven device 40 to change the orientation of the first imaging device 30a such that the center of the first image is included in the region of the face of the living body 10.

The amount of displacement is repeatedly corrected because the amount of displacement might not be corrected at one time with the calculated angle of rotation θ, by various factors including a change in occlusion caused by the three-dimensional form of the target portion 11, a change in the torque of the motor, and a deviation between the rotation axis of the motor and the optical axis of the imaging device.

When the amount of displacement is less than or equal to the threshold, the target portion 11 can fit inside the second field of view 12b. Even in this case, the following issue can arise because the size of the target portion 11 is smaller than the size of the face of the living body 10. That is, even when the amount of displacement is less than or equal to the threshold, the position of the target portion 11 in the second field of view 12b differs between before and after movement of the living body 10 as illustrated in FIG. 1A and FIG. 3B, and accurate tracking of the target portion 11 might not be possible.

In the present embodiment, in addition to a panning correction and/or a tilting correction made by the motor-driven device 40 to the imaging device 30, image-processing-based tracking based on the second image data is performed. As a result, biological information of the target portion 11 can be stably obtained before and after movement of the living body 10. An operation of the image-processing-based tracking will be described below.

### Step S106

In step S106, the processing device 50 causes the second imaging device 30b to image the target portion 11 and generate and output second image data. In the second image, an object present inside the second field of view 12b is present. The second image includes the forehead of the living body 10.

### Step S107

In step S107, the processing device 50 corrects body movement of the living body 10 by the image-processing-based tracking based on the second image data. The correction of body movement by the image-processing-based tracking is a process for limiting a displacement of the position of an image region of a portion corresponding to the target portion 11 in the second image between before and after movement of the living body 10, to less than or equal to a predetermined threshold. The predetermined threshold can be, for example, 10 pixels or three pixels. With the correction of body movement as described above, biological information of the target portion 11 can be more accurately obtained before and after movement of the living body 10.

As the correction by the image-processing-based tracking, for example, a tracking correction based on feature points of a two-dimensional image, such as the KLT algorithm, or a tracking correction by three-dimensional matching based on the ICP algorithm using a three-dimensional model by distance measurement can be applied. In the tracking correction by three-dimensional matching, in addition to corrections of displacements in the horizontal direction and the vertical direction, a three-dimensional rotation correction by a three-dimensional affine transformation is also made. As the distance measurement, a technique disclosed in, for example, International Publication No. 2021/145090 can be used. For reference, Japanese Patent Application No. 2020-005761 is hereby incorporated by reference herein in its entirety.

In the imaging system 100 according to the present embodiment, when the orientation of the imaging device 30 is changed by the motor-driven device 40, at least a portion of the forehead can be included in the second field of view 12b. When the forehead is included, the brain can be irradiated through the forehead with the light pulse emitted from the light source 20, and cerebral blood flow information can be obtained from a reflected light pulse produced by irradiation with light.

The orientation of the imaging device 30 is changed by the motor-driven device 40, and the eyebrows may be included in the second field of view 12b. When the eyebrows are included, the edge portions of the eyebrows can be used as feature points used at the time of a tracking correction, and the accuracy of a tracking correction based on the feature points of the two-dimensional image or a tracking correction by three-dimensional matching can be increased. Furthermore, the orientation of the imaging device 30 is changed by the motor-driven device 40, and the nose may be included in the second field of view 12b. When the nose is included, changes in irregularities of feature points in three-dimensional matching can be increased, and the accuracy of the tracking correction can be increased.

### Step S108

The processing device 50 determines the pixel region of the portion corresponding to the target portion 11 in the second image on the basis of the result of correction of body movement of the living body 10. The pixel region coincides with the pixel region of the portion corresponding to the target portion 11 in the second image before movement of the living body 10. In the specification, the pixel regions coinciding with each other means that a displacement between the positions of the pixel regions is less than or equal to 10 pixels. The processing device 50 generates and outputs data indicating biological information of the target portion 11 from the determined pixel region.

In the example described above with reference to FIG. 2, the amount of displacement between the center of the face and the center of the first image is used. A position other than the center of the face may be assumed to be a specific position of the face, a position other than the center of the first image is assumed to be a specific position of the first image, and the amount of displacement may be specified on the basis of the specific position of the face and the specific position of the first image. The specific position of the face can be, for example, the position of the eye or the nose. The specific position of the first image can be, for example, any one of four pixels that are respectively closest to four points of intersection of two virtual vertical lines by which the first image is divided into three equal parts in the horizontal direction and two virtual horizontal lines by which the first image is divided into three equal parts in the vertical direction.

The specific position of the first image may be determined such that a displacement between the center of the first field of view 12a and the center of the second field of view 12b can be compensated for. Such a displacement between the centers of the fields of view can be caused by a difference between the placing positions of the first imaging device 30a and the second imaging device 30b. Because of a displacement between the centers of the fields of view, even when the center of the first image is made to coincide with the center of the face of the living body 10, the target portion 11 may spread out of the second field of view 12b and the accuracy of measurement may decrease.

When the specific position of the first image is appropriately determined, a displacement between the centers of the fields of view can be compensated for, and a decrease in the accuracy of measurement can be reduced. The amount of displacement between the centers of the fields of view can be estimated by a prior calibration. The amount of displacement between the centers of the fields of view can be estimated with, for example, the following method. In this method, the first imaging device 30a and the second imaging device 30b image the same target object and obtain a first image and a second image respectively, and the coordinates of the position of the target object in the first image and those in the second image are compared with each other. Another position shifted from the center of the first image on the basis of the estimated amount of displacement between the centers of the fields of view is determined to be the specific position of the first image. When the specific position of the first image thus determined is made to coincide with the specific position of the face, a displacement between the centers of the fields of view can be compensated for, and the target portion 11 can fit inside the second field of view 12b. Furthermore, the center of the target portion 11 can be made to coincide with the center of the second image.

In the example described above with reference to FIG. 2, the second image data is generated and output when the amount of displacement between the center of the face and the center of the first image is less than or equal to the threshold. When the accuracy of measurement is not taken into consideration, the second image data may be generated and output at any timing regardless of whether the amount of displacement is less than or equal to the threshold.

In the example described above with reference to FIG. 2, the processing device 50 causes the motor-driven device 40 to change the orientations of the first imaging device 30a and the second imaging device 30b synchronously. The processing device 50 may cause the motor-driven device 40 to change the orientation of the second imaging device 30b without changing the orientation of the first imaging device 30a. For example, the processing device 50 may calculate a motion vector of the living body 10 from positional information before and after movement of the living body 10 based on the first image data and change the orientation of the second imaging device 30b by the motion vector. Example Configurations of Motor-Driven Device

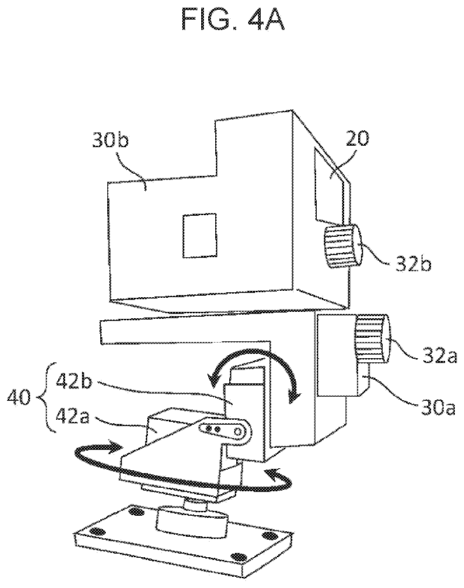

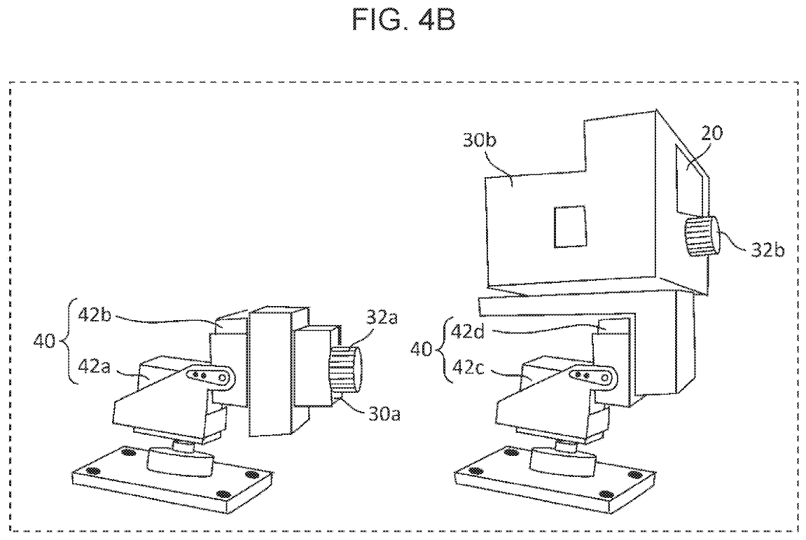

Example configurations of the motor-driven device 40 will now be described with reference to FIG. 4A to FIG. 4C. FIG. 4A is a schematic perspective view of a first example of the motor-driven device 40, which supports the imaging device 30. The motor-driven device 40 illustrated in FIG. 4A supports the first imaging device 30a and the second imaging device 30b and changes the orientation of the first imaging device 30a and the orientation of the second imaging device 30b synchronously. To the second imaging device 30b, the light source 20 is attached.

The motor-driven device 40 illustrated in FIG. 4A includes a first motor-driven mechanism 42a and a second motor-driven mechanism 42b for making a panning correction and a tilting correction to the imaging device 30 respectively. The first imaging device 30a includes a first lens 32a having a relatively wide field of view, and the second imaging device 30b includes a second lens 32b having a relatively narrow field of view. The first field of view 12a and the second field of view 12b illustrated in FIG. 1A are determined by the first lens 32a and the second lens 32b respectively. The first imaging device 30a and the second imaging device 30b are disposed such that the first lens 32a and the second lens 32b come close to each other. With such disposition, the center of the field of view of the first lens 32a and the center of the field of view of the second lens 32b can be made to come close to each other. When the orientation of the first imaging device 30a and the orientation of the second imaging device 30b are changed synchronously, the center position of the face in the first image can be corrected and the center position of the target portion 11 in the second image can be corrected at the same time.

The distance between the optical axes of the first lens 32a and the second lens 32b can be, for example, less than or equal to 80 mm. At this time, in a configuration in which the distance between the center of the second lens 32b and the center of the target portion 11 is 50 cm, with reference to the center of the first lens 32a or the center of the second lens 32b, the angle of displacement between the center of the first field of view 12a and the center of the second field of view 12b can be limited to less than or equal to 10°. Furthermore, when the distance between the optical axes of the first lens 32a and the second lens 32b is, for example, less than or equal to 40 mm, the above-described angle of displacement can be limited to less than or equal to 5°. When the distance between the optical axes of the first lens 32a and the second lens 32b is, for example, less than or equal to 20 mm, the above-described angle of displacement can be limited to less than or equal to 3º.

FIG. 4B is a schematic perspective view of a second example of the motor-driven device 40, which supports the imaging device 30. The motor-driven device 40 illustrated in FIG. 4B includes the first motor-driven mechanism 42a and the second motor-driven mechanism 42b for making a panning correction and a tilting correction to the first imaging device 30a respectively. The motor-driven device 40 illustrated in 4B further includes a third motor-driven mechanism 42c and a fourth motor-driven mechanism 42d for making a panning correction and a tilting correction to the second imaging device 30b respectively. The motor-driven device 40 illustrated in 4B can change the orientation of the first imaging device 30a and the orientation of the second imaging device 30b separately. Therefore, the orientation of the second imaging device 30b can be changed without changing the orientation of the first imaging device 30a.

The motor-driven device 40 illustrated in 4B can be designed such that the optical axis of the first lens 32a is made to come close to the rotation axes of the first motor-driven mechanism 42a and the second motor-driven mechanism 42b and the optical axis of the second lens 32b is made to come close to the rotation axes of the third motor-driven mechanism 42c and the fourth motor-driven mechanism 42d. When the optical axis of each lens is made to come close to the rotation axes of the corresponding motor-driven mechanisms, the accuracy of estimation of the amount of rotation of panning rotation and/or tilting rotation in step S104 can be increased, and the number of times the amount of displacement is repeatedly corrected can be decreased.

FIG. 4C is a schematic perspective view of a third example of the motor-driven device 40, which supports the imaging device 30. The motor-driven device 40 illustrated in FIG. 4C includes an arm structure that can change the orientation of the imaging device 30 in six-axis directions. The six-axis directions include the front-back direction, the up-down direction, the left-right direction, the panning direction, the tilting direction, and the rolling direction. With the motor-driven device 40 illustrated in FIG. 4C, the positional relationship between the second imaging device 30b and the target portion 11 can be more accurately corrected. Specifically, the second imaging device 30b can be moved in the range directions, and even when the target portion 11 approaches the second imaging device 30b or moves away from the second imaging device 30b, the distance between the second imaging device 30b and the target portion 11 can be kept constant. As a result, even when the living body 10 moves with a higher degree of freedom, biological information of the target portion 11 can be stably obtained.

### Modification

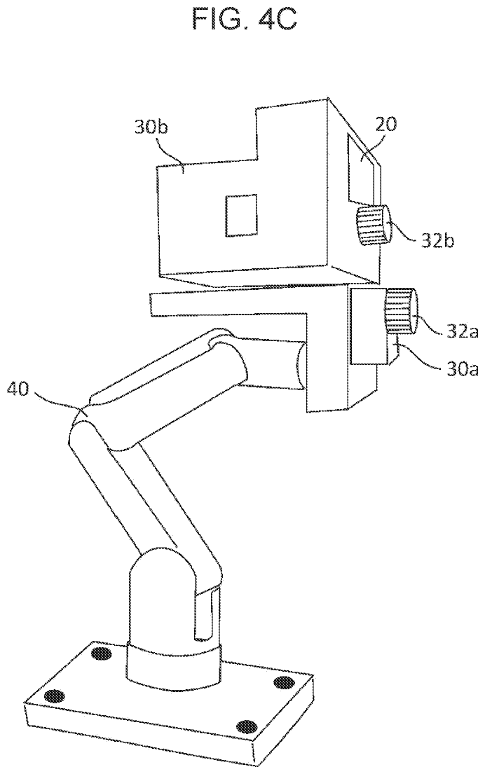

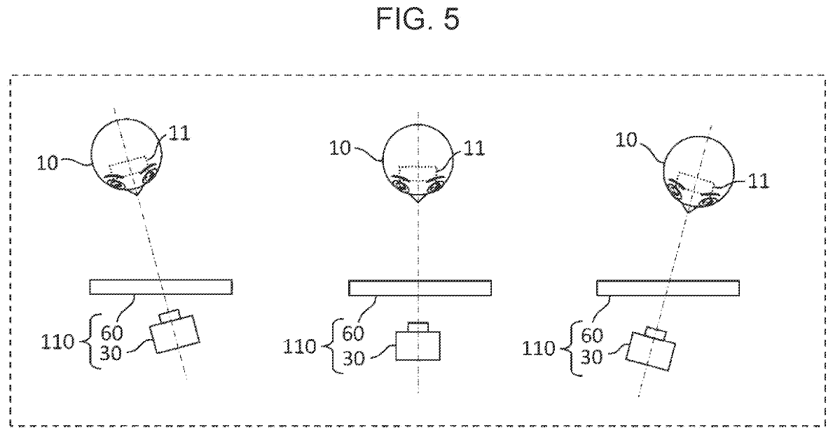

A modification of the imaging system 100 according to the present embodiment will now be described with reference to FIG. 5. FIG. 5 is a diagram schematically illustrating examples of imaging of the living body 10 by an imaging system according to the modification of the present embodiment. An imaging system 110 illustrated in FIG. 5 includes a display 60 in addition to the configurations of the imaging system 100 illustrated in FIG. 1A. Among the configurations of the imaging system 100 illustrated in FIG. 1, configurations other than the imaging device 30 are omitted in FIG. 5. In the example illustrated in FIG. 5, the living body 10 is viewing the display 60 from the front, the right-hand side, or the left-hand side of the display 60 when viewed from the living body 10. The display 60 is disposed in the vicinity of the imaging device 30 but not between the living body 10 and the imaging device 30. The vicinity means that the minimum distance of the gap between the display 60 and an imaging device closer to the display 60 among the first imaging device 30a and the second imaging device 30b is less than or equal to 50 cm. In the example illustrated in FIG. 5, the imaging device 30 is at a position behind the back side of the display 60 and higher than the display 60. The imaging device 30 can be disposed at, for example, any of positions above, below, to the left, and to the right of the display 60 when viewed from the living body 10. The display 60 can be, for example, a monitor of a desktop PC, a monitor of a laptop PC, or a monitor of an examination device.

In this modification, regardless of a direction in which the living body 10 is viewing the display 60, biological information of the target portion 11 can be obtained in a state in which the imaging device 30 is always made to face the living body 10, by making a panning correction and/or a tilting correction to the imaging device 30. When the living body 10 views the display 60, an angle made by the optical axis of the second imaging device 30b and the forehead surface of the target portion 11 is always kept constant. The intensity of incidence when the light pulse emitted from the light source 20 is incident on the forehead surface of the target portion 11 depends on the angle of incidence. Therefore, keeping constant the angle made by the optical axis of the second imaging device 30b and the forehead surface of the target portion 11 is effective in stably obtaining biological information of the target portion 11.

Furthermore, a function of detecting the orientation of the face of the living body 10 may be added to the imaging device 30. The orientation of the face of the living body 10 is the orientation of the face to the imaging device 30 or the display 60. The processing device 50 may detect the orientation of the face on the basis of the first image data and/or the second image data, and when the face of the living body 10 is oriented in the direction of the imaging device 30 or the display 60, may generate and output biological information of the target portion 11. The processing device 50 may further utilize the generated biological information in, for example, estimation of the mental condition and/or the physical condition of the living body 10. That is, the processing device 50 may determine whether to generate and output biological information and whether to utilize the biological information, on the basis of the detected orientation of the face. For example, when the amount of displacement between the specific position of the face of the living body 10 and the specific position of the first image exceeds a specific threshold, the processing device 50 may restrict generation and output of biological information. With such a restriction, when the living body 10 looks away or is away from their desk, noise data different from data of biological information to be obtained can be excluded. As a method for detecting the orientation of the face, for example, a method of estimating the orientation of the face by landmark detection in which feature points, such as the eyes, nose, mouth, and profile of the face, are detected or a method of estimating the orientation of the face from three-dimensional data of the face may be used.

### Working Example

A working example of the imaging system 100 according to the present embodiment will now be described together with a comparative example. In the working example, cerebral blood flow information of the target portion 11 after movement was obtained after the orientation of the imaging device 30 was changed in accordance with movement of the living body 10. In contrast, in the comparative example, cerebral blood flow information of the target portion 11 after movement was obtained in a state in which the orientation of the imaging device 30 was fixed.

In the working example and the comparative example, as the living body 10, a phantom model that is an imitation of a human head was irradiated with a near-infrared light pulse. The absorption coefficient and the scattering coefficient of the phantom model are equal to the absorption coefficient and the scattering coefficient of human heads respectively. To reproduce movement of the living body 10, the imaging system 100 was moved by a driving stage to thereby change the relative positions of the imaging device 30 and the phantom model. The driving stage can move the imaging system 100 in an X direction and/or a Y direction. The X direction and the Y direction are the horizontal direction and the vertical direction of the first image respectively. The amount of movement of the living body 10 was set to ±10 mm, ±20 mm, ±30 mm, ±60 mm, and ±90 mm in the X direction and ±10 mm, ±20 mm, and ±30 mm in the Y direction. Although the amount of movement of the living body 10 may be further larger, the amount of movement of the living body 10 was within a range with which the target portion 11 is included in the second field of view 12b so as to allow a comparison between the working example in which a panning correction and/or a tilting correction is made to the imaging device 30 and the comparative example in which such a correction is not made. The orientation of the first imaging device 30a and the orientation of the second imaging device 30b were changed synchronously by the motor-driven device 40 illustrated in FIG. 4A.

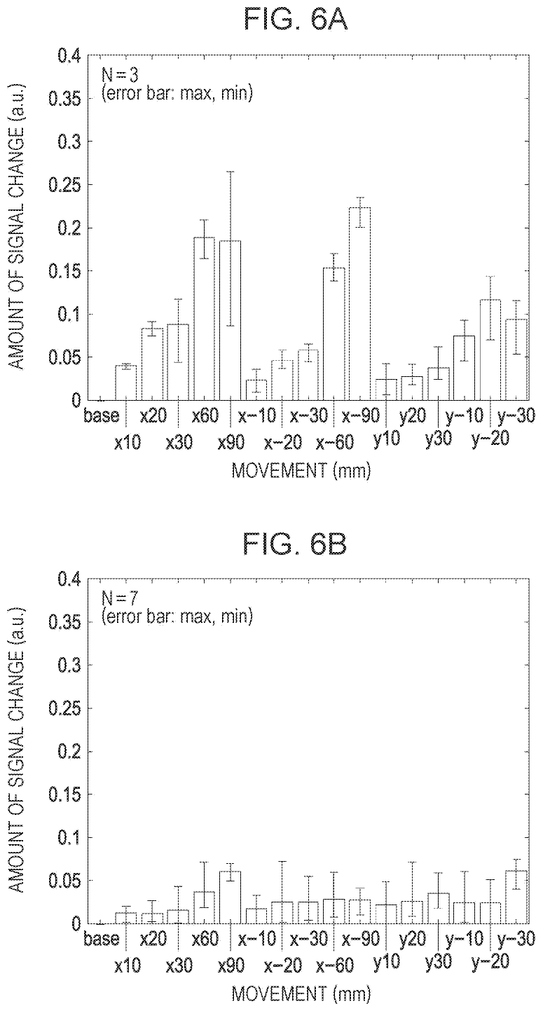

FIG. 6A is a diagram illustrating the comparative example in which cerebral blood flow information of the target portion 11 after movement was obtained in a state in which the orientation of the imaging device 30 was fixed. FIG. 6B is a diagram illustrating the working example in which cerebral blood flow information of the target portion 11 after movement was obtained after the orientation of the imaging device 30 was changed in accordance with movement of the living body 10. In each of FIG. 6A and FIG. 6B, the horizontal axis represents the amount of movement (mm) of the living body 10, and the vertical axis represents the amount of signal change, from an initial value, obtained from the second image data. “base” on the horizontal axis represents the initial state before movement. In the working example and the comparative example, a software-based tracking correction by three-dimensional matching based on the face form of the phantom was made, and the amount of signal change from the initial value in a central region of the forehead, which is an ROI (Region of Interest), was subsequently measured. The number of times measurement was performed is three in the comparative example and seven in the working example. The size of each of the bars illustrated in FIG. 6A and FIG. 6B represents the average of the absolute values of measured amounts of signal changes. Each of the error bars represents a range from the minimum value to the maximum value of the absolute value of the measured amount of signal change. The amount of signal change may be zero because of no change in the cerebral blood flow before and after movement of the living body 10.

In the comparative example illustrated in FIG. 6A, as the amount of movement of the living body 10 increased, the amount of signal change changed to a larger degree. A possible factor of the changes in the signal value is that with big movement, an error in the tracking correction by three-dimensional matching increased and an error in the illuminance distribution of an illuminance light pulse increased in the ROI. In contrast, in the working example illustrated in FIG. 6B, the absolute values of the amounts of signal changes were mostly small and were decreased to about ¼ to ½ of those in the comparative example illustrated in FIG. 6A. Even when the amount of movement of the living body 10 is 90 mm, a significant improvement from the comparative example illustrated in FIG. 6A was seen.

From the above, it has been found that the following effects are attained by the imaging system 100 according to the present embodiment. The target portion 11 of the living body 10 after movement can be included in the second field of view 12b, and furthermore, the accuracy of the tracking correction by 3D matching can be increased and an error in the illuminance distribution of the illuminance light pulse can be decreased. As a result, even when the living body 10 moves, biological information can be stably obtained.

In the working example, a panning correction and/or a tilting correction was made to the imaging device 30 so as to allow the imaging device 30 to follow the living body 10 that moves in the X direction and/or the Y direction. When a further correction is made to the imaging device 30 so as to allow the imaging device 30 to follow the living body 10 that also moves in a Z direction perpendicular to the X direction and the Y direction, it is expected that biological information can be more stably obtained.

Matters regarding obtaining of internal information of the target portion 11 will be described below. The matters include a configuration of the second imaging device 30b, an operation of emitting the first light pulse Iₚ₁ and the second light pulse Iₚ₂, a method for detecting the internal scattered component I₂, and calculation of the amount of change in the concentration of each of HbO₂ and Hb in blood from an initial value.

### Configuration of Second Imaging Device 30b

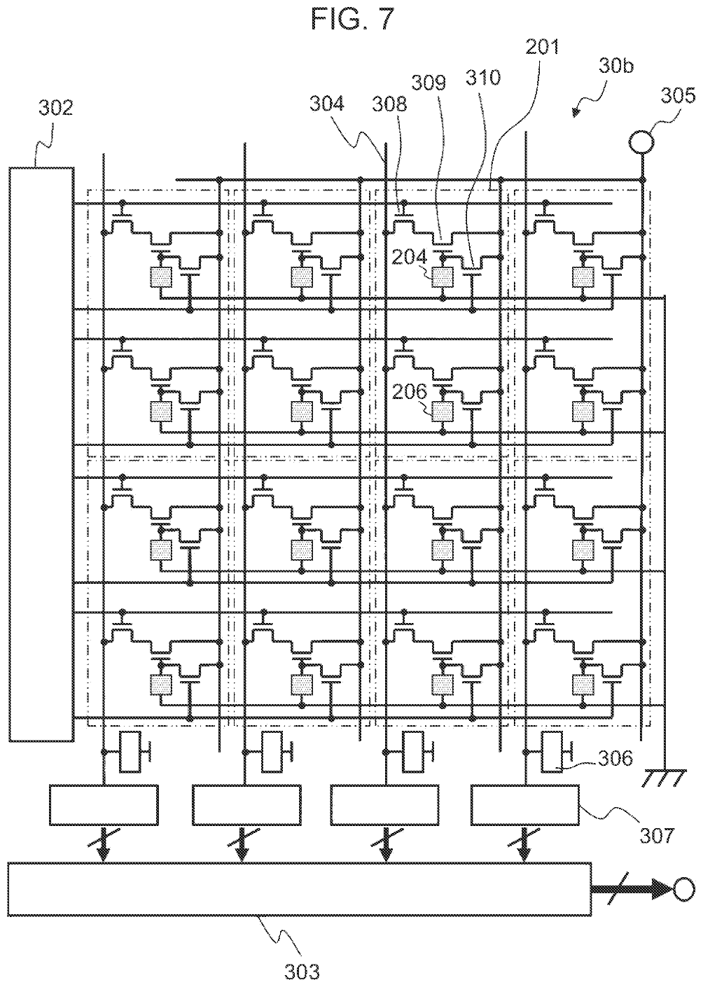

An example configuration of the second imaging device 30b will now be described with reference to FIG. 7. FIG. 7 is a diagram illustrating an example configuration of the second imaging device 30b. In FIG. 7, the region enclosed within a frame of a two-dot chain line corresponds to one pixel 201. The pixel 201 includes one photodiode not illustrated. Although FIG. 7 illustrates eight pixels arranged in two rows and four columns, a larger number of pixels can be actually disposed. Each pixel 201 includes a first floating diffusion layer 204 and a second floating diffusion layer 206. It is assumed here that the wavelength of the first light pulse Iₚ₁ is greater than or equal to 650 nm and shorter than 805 nm and the wavelength of the second light pulse Iₚ₂ is longer than 805 nm and less than or equal to 950 nm. The first floating diffusion layer 204 stores a charge produced by receiving a first reflected-light pulse resulting from the first light pulse Iₚ₁. The second floating diffusion layer 206 stores a charge produced by receiving a second reflected-light pulse resulting from the second light pulse Iₚ₂. Signals stored in the first floating diffusion layer 204 and the second floating diffusion layer 206 are handled as if the signals are signals of two pixels of a typical CMOS image sensor and are output from the second imaging device 30b.

Each pixel 201 has two signal detection circuits. Each signal detection circuit includes a source follower transistor 309, a row selection transistor 308, and a reset transistor 310. Each transistor is, for example, a field-effect transistor formed on a semiconductor substrate but is not limited to this. As illustrated, one of the input terminal and the output terminal of the source follower transistor 309 and one of the input terminal and the output terminal of the row selection transistor 308 are connected. The one of the input terminal and the output terminal of the source follower transistor 309 is typically the source. The one of the input terminal and the output terminal of the row selection transistor 308 is typically the drain. The gate of the source follower transistor 309, which is a control terminal, is connected to the photodiode. A signal charge of a hole or an electron generated by the photodiode is stored in the floating diffusion layer, which is the charge storage unit between the photodiode and the source follower transistor 309.

Although not illustrated in FIG. 7, the first floating diffusion layer 204 and the second floating diffusion layer 206 are connected to the photodiode. Between the photodiode and each of the first floating diffusion layer 204 and the second floating diffusion layer 206, a switch can be provided. This switch switches a conduction state between the photodiode and each of the first floating diffusion layer 204 and the second floating diffusion layer 206 in accordance with a signal storage pulse from the processing device 50. Accordingly, a start and a stop of storing of a signal charge in each of the first floating diffusion layer 204 and the second floating diffusion layer 206 are controlled. The electronic shutter in the present embodiment has a mechanism for such exposure control.

The signal charges stored in the first floating diffusion layer 204 and the second floating diffusion layer 206 are read in response to the gate of the row selection transistor 308 being turned ON by a row selection circuit 302. At this time, a current flowing into the source follower transistor 309 and a source follower load 306 from a source follower power supply 305 is amplified in accordance with the signal potentials of the first floating diffusion layer 204 and the second floating diffusion layer 206. An analog signal based on this current read from a vertical signal line 304 is converted to digital signal data by an analog-digital (AD) conversion circuit 307 connected on a per column basis. This digital signal data is read by a column selection circuit 303 on a per column basis and output from the second imaging device 30b. The row selection circuit 302 and the column selection circuit 303 perform reading from one row, subsequently perform reading from the next row, and read information of the signal charges in the floating diffusion layers of every row in a similar manner. After reading of all signal charges, the processing device 50 turns on the gate of each reset transistor 310 to thereby reset all floating diffusion layers. Accordingly, imaging for one frame is completed. High-speed imaging for a frame is repeated in a similar manner, and imaging for a series of frames by the second imaging device 30b is completed.

Although an example in which the second imaging device 30b is of CMOS type has been described in the present embodiment, the second imaging device 30b may be another type of imaging element. The second imaging device 30b may be, for example, of CCD type, a single photon counting element, or an amplification image sensor, such as an EMCCD or ICCD.

Operation of Emitting First Light Pulse Iₚ₁ and Second Light Pulse Iₚ₂

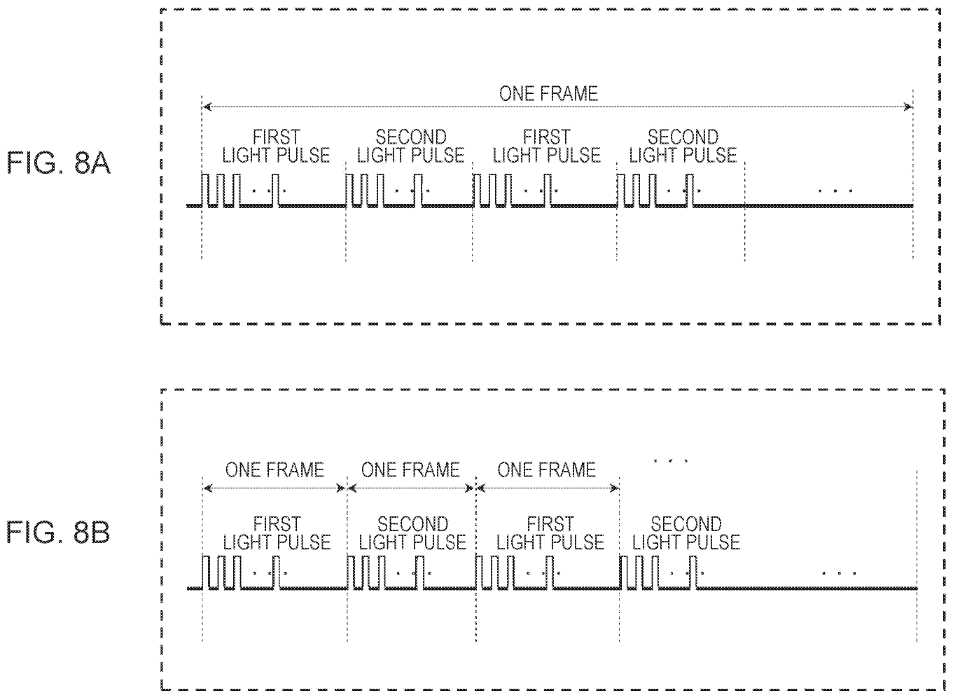

An operation of emitting the first light pulse Iₚ₁ and the second light pulse Iₚ₂ will now be described with reference to FIG. 8A and FIG. 8B. FIG. 8A is a diagram illustrating an example operation of emitting the first light pulse Iₚ₁ and the second light pulse Iₚ₂. As illustrated in FIG. 8A, switching between emission of the first light pulse Iₚ₁ and emission of the second light pulse Iₚ₂ may be performed multiple times within one frame. As a result, the time difference between the timings when detection images based on two types of wavelengths are obtained can be reduced, and even when the target portion 11 moves, imaging by using the first light pulse Iₚ₁ and imaging by using the second light pulse Iₚ₂ can be performed almost at the same time

FIG. 8B is a diagram illustrating another example operation of emitting the first light pulse Iₚ₁ and the second light pulse Iₚ₂. As illustrated in FIG. 8B, switching between emission of the first light pulse Iₚ₁ and emission of the second light pulse Iₚ₂ may be performed for each frame. As a result, switching between detection of a reflected-light pulse resulting from the first light pulse Iₚ₁ and detection of a reflected-light pulse resulting from the second light pulse Iₚ₂ can be performed for each frame. In this case, each pixel 201 may include a single charge storage unit. With such a configuration, the number of charge storage units in each pixel 201 can be reduced, and therefore, the size of each pixel 201 can be increased, and the sensitivity can be increased.

### Method for Detecting Internal Scattered Component I₂

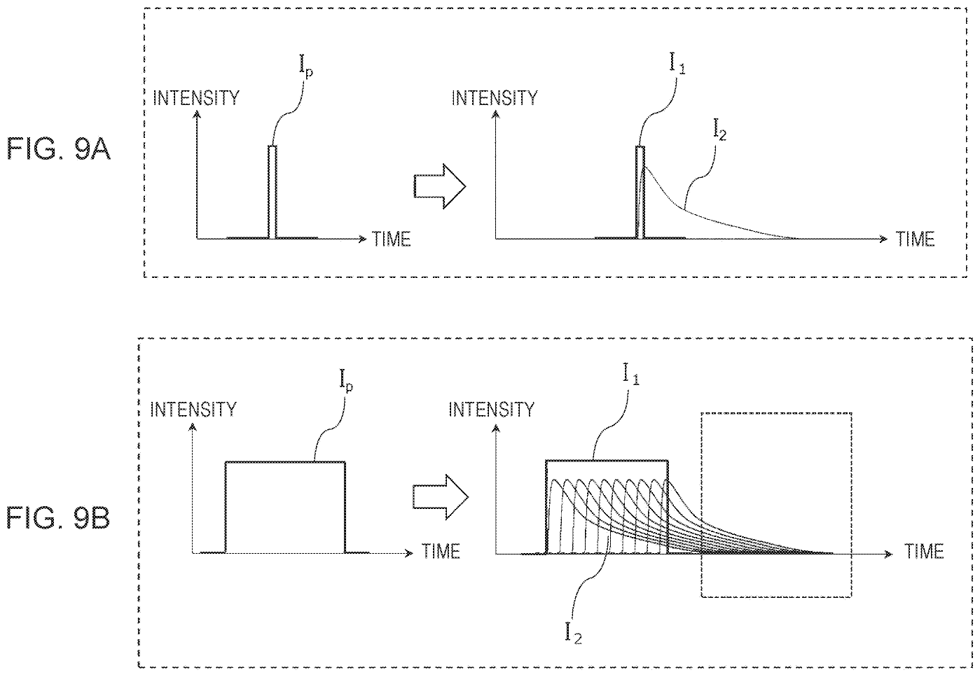

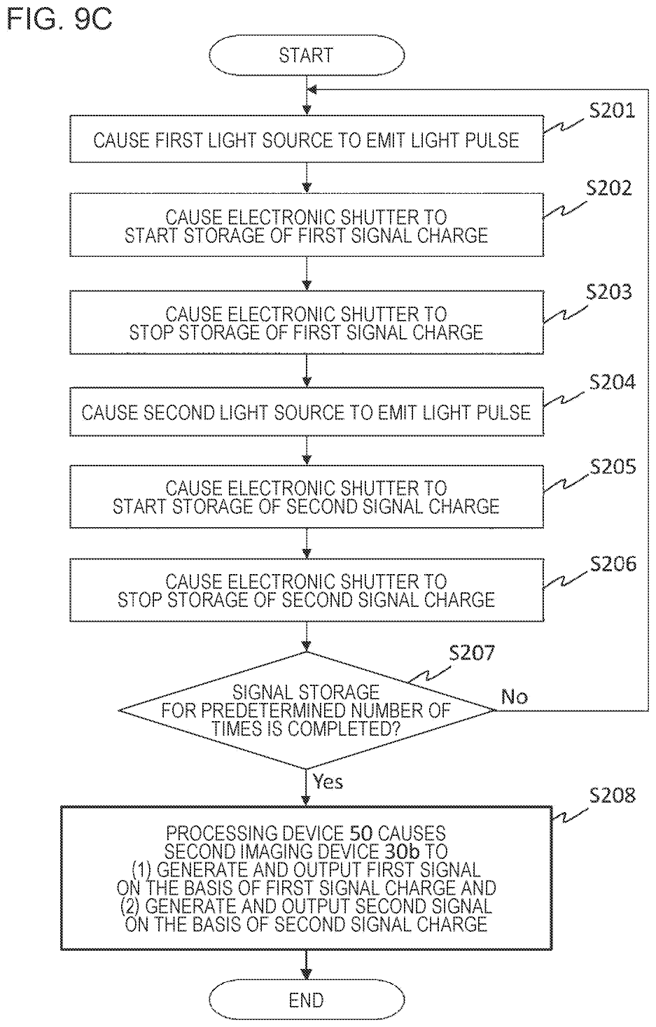

A method for detecting the internal scattered component I₂ will be described below with reference to FIG. 9A to FIG. 9C.

FIG. 9A is a diagram schematically illustrating example changes over time of the surface-reflected component I₁ and the internal scattered component I₂ included in a reflected-light pulse when the light pulse Iₚ has an impulse waveform. FIG. 9B is a diagram schematically illustrating example changes over time of the surface-reflected component I₁ and the internal scattered component I₂ included in a reflected-light pulse when the light pulse Iₚ has a rectangular waveform. The left-hand diagram in each of FIG. 9A and FIG. 9B represents an example waveform of the light pulse Iₚ emitted from the light source 20, and the right-hand diagram therein represents example waveforms of the surface-reflected component I₁ and the internal scattered component I₂ included in the reflected-light pulse.

When the light pulse Iₚ has an impulse waveform, the surface-reflected component I₁ has a waveform similar to that of the light pulse Iₚ, and the internal scattered component I₂ has an impulse response waveform that lags behind the surface-reflected component I₁, as shown by the right-hand diagram in FIG. 9A. This is because the internal scattered component I₂ corresponds to a combination of light rays that have passed through various paths in the target portion 11.

When the light pulse Iₚ has a rectangular waveform, the surface-reflected component I₁ has a waveform similar to that of the light pulse Iₚ, and the internal scattered component I₂ has a waveform formed of superimposed impulse response waveforms, as shown by the right-hand diagram in FIG. 9B. The present inventors have confirmed that when impulse response waveforms are superimposed, the amount of light of the internal scattered component I₂ detected by the imaging device 30 can be amplified compared to a case where the light pulse Iₚ has an impulse waveform. When the electronic shutter is started in a falling portion of the reflected-light pulse, the internal scattered component I₂ can be effectively detected. The region outlined by a dashed line in the right-hand diagram in FIG. 9B indicates an example shutter open period during which the electronic shutter of the imaging device 30 is opened. When the pulse width of the rectangular pulse is of the order of 1 ns to 10 ns, the light source 20 can be driven at a low voltage. This can reduce the size and costs of the imaging system 100 in the present embodiment.

To detect information about the coefficients of light absorption and the coefficients of light scattering at different locations inside a living body in the depth direction in a distinguished manner, a streak camera has been used. For example, Japanese Unexamined Patent Application Publication No. 4-189349 discloses an example of such a streak camera. In the streak camera, for measurement with a desired spatial resolution, an ultrahigh frequency light pulse having a femtosecond or picosecond pulse width is used. In contrast, in the present embodiment, the surface-reflected component I₁ and the internal scattered component I₂ can be detected in a distinguished manner. Therefore, the light pulse emitted from the light source 20 need not be an ultrahigh frequency light pulse, and any pulse width can be selected.

When the head of the living body 10 is irradiated with light to measure the cerebral blood flow, the amount of light of the internal scattered component I₂ can have a very small value that is equal to about several thousandths to several tens of thousandths of the amount of light of the surface-reflected component I₁. Furthermore, taking into consideration the laser safety standard, the amount of light that can be used in irradiation is very small. Therefore, detection of the internal scattered component I₂ becomes very difficult. In this case, when the light source 20 emits the light pulse Iₚ having a relatively large pulse width, the total amount of the internal scattered component I₂ having a time lag can be increased. This can increase the amount of detected light and improve the SN ratio.

The light source 20 can emit, for example, the light pulse Iₚ having a pulse width of greater than or equal to 3 ns. Alternatively, the light source 20 may emit the light pulse Iₚ having a pulse width of greater than or equal to 5 ns or greater than or equal to 10 ns. When the pulse width is too large, non-used light increases, which is wasteful, and therefore, the light source 20 can emit, for example, the light pulse Iₚ having a pulse width of less than or equal to 50 ns. Alternatively, the light source 20 may emit the light pulse Iₚ having a pulse width of less than or equal to 30 ns or less than or equal to 20 ns. When the pulse width of the rectangular pulse is several nanoseconds to several tens of nanoseconds, the light source 20 can be driven at a low voltage. This can reduce costs of the imaging system 100 in the present embodiment.

The irradiation pattern of the light source 20 may be, for example, a pattern having a uniform intensity distribution within an irradiation region. In this regard, the imaging system 100 in the present embodiment is different from an existing apparatus disclosed in, for example, Japanese Unexamined Patent Application Publication No. 11-164826. With the apparatus disclosed in Japanese Unexamined Patent Application Publication No. 11-164826, a detector and a light source is spaced apart from each other by about 3 cm, and a surface-reflected component is spatially separated from an internal scattered component, and therefore, the irradiation pattern is to be a pattern having a discrete intensity distribution. In contrast, in the present embodiment, the surface-reflected component I₁ can be separated from the internal scattered component I₂ in terms of time and reduced. Therefore, the light source 20 that has an irradiation pattern having a uniform intensity distribution can be used. The irradiation pattern having a uniform intensity distribution may be formed by diffusing light emitted from the light source 20 with a diffuser plate.

Unlike in the related art, the internal scattered component I₂ directly under an irradiation point of the target portion 11 can also be detected in the present embodiment. When the target portion 11 is irradiated with light across a spatially wide area, the resolution of measurement can be increased.

FIG. 9C is a flowchart schematically illustrating operations of the processing device 50 performed in relation to the first light source 20a, the second light source 20b, and the second imaging device 30b. The processing device 50 performs the operations schematically illustrated in FIG. 9C to thereby cause the second imaging device 30b to detect at least a component of each of the first and second reflected-light pulses in a falling period.

### Step S201

In step S201, the processing device 50 causes the first light source 20a to emit the first light pulse Iₚ₁ for a predetermined time. At this time, the electronic shutter of the second imaging device 30b is in a state in which exposure is stopped. The processing device 50 keeps the electronic shutter in the state in which exposure is stopped, until the end of a period in which the surface-reflected component I₁ in the first reflected-light pulse reaches the second imaging device 30b.

### Step S202

In step S202, the processing device 50 causes the electronic shutter to start exposure at the timing when the internal scattered component I₂ in the first reflected-light pulse reaches the second imaging device 30b.

### Step S203

In step S203, the processing device 50 causes the electronic shutter to stop exposure after a lapse of a predetermined time. As a result of steps S202 and S203, a signal charge is stored in the first floating diffusion layer 204 illustrated in FIG. 7. This signal charge is referred to as “first signal charge”.

### Step S204

In step S204, the processing device 50 causes the second light source 20b to emit the second light pulse Iₚ₂ for a predetermined time. At this time, the electronic shutter of the second imaging device 30b is in a state in which exposure is stopped. The processing device 50 keeps the electronic shutter in the state in which exposure is stopped, until the end of a period in which the surface-reflected component I₁ in the second reflected-light pulse reaches the second imaging device 30b.

### Step S205

In step S205, the processing device 50 causes the electronic shutter to start exposure at the timing when the internal scattered component I₂ in the second reflected-light pulse reaches the second imaging device 30b.

### Step S206

In step S206, the processing device 50 causes the electronic shutter to stop exposure after a lapse of a predetermined time. As a result of steps S205 and S206, a signal charge is stored in the second floating diffusion layer 206 illustrated in FIG. 7. This signal charge is referred to as “second signal charge”.

### Step S207

In step S207, the processing device 50 determines whether the number of times the above-described signal storage is performed reaches a predetermined number of times. If determination in step S207 results in No, the processing device 50 repeats step S201 to step S206 until the determination results in Yes. If determination in step S207 results in Yes, the processing device 50 performs the operation in step S208.

### Step S208

In step S208, the processing device 50 causes the second imaging device 30b to generate and output a first signal on the basis of the first signal charge, and the processing device 50 causes the second imaging device 30b to generate and output a second signal on the basis of the second signal charge. The first signal and the second signal reflect internal information of the target portion 11.

The operations illustrated in FIG. 9C are summarized as follows. The processing device 50 performs a first operation of causing the first light source 20a to emit the first light pulse Iₚ₁ and causing the second imaging device 30b to detect at least a component of the first reflected-light pulse in the falling period. The processing device 50 performs a second operation of causing the second light source 20b to emit the second light pulse Iₚ₂ and causing the second imaging device 30b to detect at least a component of the second reflected-light pulse in the falling period. The processing device 50 repeats a series of operations including the first operation and the second operation a predetermined number of times. Alternatively, the processing device 50 may repeat the first operation a predetermined number of times and subsequently repeat the second operation a predetermined number of times. The first operation and the second operation may be interchanged.

With the operations illustrated in FIG. 9C, the internal scattered component I₂ can be detected with high sensitivity. When the head of the living body 10 is irradiated with light to obtain internal information of, for example, the cerebral blood flow, the attenuation rate of light inside is very large. For example, outgoing light can be attenuated to about one millionth of the incident light. Therefore, with one-pulse irradiation, the amount of light may be insufficient to detect the internal scattered component I₂. With irradiation based on Class 1 of the laser safety standard, the amount of light is specifically very small. In this case, when the light source 20 emits the light pulse multiple times and the second imaging device 30b performs exposure with the electronic shutter multiple times, detection signals can be added up and the sensitivity can be increased. Light emission and exposure need not be performed multiple times and are performed as necessary.

In the above-described example, when the second imaging device 30b is caused to detect at least a component of each of the first and second reflected-light pulses in the rising period, the surface-reflected component I₁ of each of the first and second reflected-light pulses can be detected, and surface information of, for example, the face or scalp blood flow can be obtained. The first floating diffusion layer 204 included in each pixel 201 illustrated in FIG. 7 can store a charge produced by receiving at least a component of the first reflected-light pulse in the rising period, and the second floating diffusion layer 206 included therein can store a charge produced by receiving at least a component of the second reflected-light pulse in the rising period.

Two pixels 201 adjacent to each other in the row direction illustrated in FIG. 7 may be handled as one pixel. For example, the first floating diffusion layer 204 included in one of the pixels 201 can store a charge produced by receiving at least a component of the first reflected-light pulse in the falling period, and the second floating diffusion layer 206 included therein can store a charge produced by receiving at least a component of the second reflected-light pulse in the falling period. The first floating diffusion layer 204 included in the other pixel 201 can store a charge produced by receiving at least a component of the first reflected-light pulse in the rising period, and the second floating diffusion layer 206 included therein can store a charge produced by receiving at least a component of the second reflected-light pulse in the rising period. With such a configuration, internal information and surface information of the living body 10 can be obtained.

Calculation of Amount of Change in Concentration of Each of HbO₂ and Hb in Blood from Initial Value

When the first wavelength of the light pulse Iₚ₁ is greater than or equal to 650 nm and shorter than 805 nm and the second wavelength of the second light pulse Iₚ₂ is longer than 850 nm and less than or equal to 950 nm, the amount of change in the concentration of each of HbO₂ and Hb in blood from an initial value can be obtained by solving predetermined simultaneous equations by using the first signal and the second signal. Equation (1) and equation (2) below are examples of the simultaneous equations.

$${ε}_{OXY}^{750} Δ {HbO}_{2} + {ε}_{deOXY}^{750} Δ Hb = - ln \frac{{I}_{now}^{750}}{{I}_{ini}^{750}} \qquad ( 1 )$$

$${ε}_{OXY}^{850} Δ {HbO}_{2} + {ε}_{deOXY}^{850} Δ Hb = - ln \frac{{I}_{now}^{850}}{{I}_{ini}^{850}} \qquad ( 2 )$$

ΔHbO₂ and ΔHb respectively denote the amounts of changes in the concentrations of HbO₂ and Hb in blood from respective initial values. ε⁷⁵⁰_{OXY} and ε⁷⁵⁰_{deOXY} deoxy respectively denote the molar absorption coefficients of HbO₂ and Hb at a wavelength 750 nm. ε⁸⁵⁰_{OXY} and ε⁸⁵⁰_{deOXY} deoxy respectively denote the molar absorption coefficients of HbO₂ and Hb at a wavelength 850 nm. I⁷⁵⁰ᵢₙᵢ and I⁷⁵⁰_{now} respectively denote detection intensities at a reference time (initial time) and a specific time at a wavelength 750 nm. These symbols represent, for example, detection intensities in a state in which the brain is not activated and in a state in which the brain is activated. I⁸⁵⁰ᵢₙᵢ and I⁸⁵⁰_{now} respectively denote detection intensities at the reference time (initial time) and the specific time at a wavelength 850 nm. These symbols represent, for example, detection intensities in a state in which the brain is not activated and in a state in which the brain is activated.

The process illustrated by the flowchart in FIG. 9C may be performed one time before an examinee experiences a specific event A, and the process illustrated by the flowchart in FIG. 9C may be performed one time after the examinee has experienced the specific event A. In this case, variables in equations (1) and (2) above may be defined as follows.
I⁷⁵⁰ᵢₙᵢ=(the intensity of the first signal generated by the second imaging device 30b on the basis of the first reflected light pulse corresponding to the first light pulse emitted by the first light source 20a toward the examinee before the examinee experiences the specific event A)
I⁸⁵⁰ᵢₙᵢ=(the intensity of the second signal generated by the second imaging device 30b on the basis of the second reflected light pulse corresponding to the second light pulse emitted by the second light source 20b toward the examinee before the examinee experiences the specific event A)
I⁷⁵⁰_{now}=(the intensity of the first signal generated by the second imaging device 30b on the basis of the first reflected light pulse corresponding to the first light pulse emitted by the first light source 20a toward the examinee after the examinee has experienced the specific event A)
I⁸⁵⁰_{now}=(the intensity of the second signal generated by the second imaging device 30b on the basis of the second reflected light pulse corresponding to the second light pulse emitted by the second light source 20b toward the examinee after the examinee has experienced the specific event A)
ΔHbO₂={(the concentration of HbO₂ in blood of the examinee after the examinee has experienced the specific event A)−(the concentration of HbO₂ in blood of the examinee before the examinee experiences the specific event A)}
ΔHb={(the concentration of Hb in blood of the examinee after the examinee has experienced the specific event A)−(the concentration of Hb in blood of the examinee before the examinee experiences the specific event A)}

### Other Matter 1

The processes in S102 to S105 illustrated in FIG. 2 may be processes in S102′ to S105′ described below. These processes will be described with reference to FIG. 3C for explaining an amount of displacement Q1 and an amount of displacement Q2 in the first image and FIG. 3D for explaining a first amount of rotation and a second amount of rotation.

Step S102′ (a Process that is an Alternative to Step S102)

The processing device 50 extracts a face region 112 that includes the face of the living body 10 from a first image 112a by a machine learning process on the basis of the first image data and calculates the amount of displacement between a center O112 of the face region and a center O112a of the first image 112a. The amount of displacement includes the amount of displacement Q1, which is the amount of displacement in the horizontal direction, and the amount of displacement Q2, which is the amount of displacement in the vertical direction (see FIG. 3C).

The processing device 50 includes a cascade classifier (not illustrated) that has learned human faces. The cascade classifier reads the first image data and outputs information for identifying the face region 112 that includes the face of the living body 10 in the first image 112a (for example, the two-dimensional coordinates of each of the four corners of the frame of the face region 112).

Step S103′ (a Process that is an Alternative to Step S103)

The processing device 50 performs first determination as to whether the amount of displacement Q1 is less than or equal to a first threshold and/or second determination as to whether the amount of displacement Q2 is less than or equal to a second threshold. The first threshold may be a value equal to ½ of a breath Q3 of the face region 112, and the second threshold may be a value equal to ½ of a length Q4 of the face region 112. If the first determination results in Yes or the second determination results in Yes, the processing device 50 performs the operation in step S106. If the first determination results in No and the second determination results in No, the processing device 50 performs the operation in step S104′.

Step S104′ (a Process that is an Alternative to Step S104)

The processing device 50 determines the first amount of rotation of panning rotation of the motor-driven device 40 and the second amount of rotation of tilting rotation of the motor-driven device 40.

Each of the first amount of rotation and the second amount of rotation is determined on the basis of the three-dimensional coordinates (x1, y1, z1) of a first point corresponding to the center O112 of the face region 112 (see FIG. 3D). The three-dimensional coordinates (x1, y1, z1) may be determined by providing the imaging device 30 with a stereo camera (stereo camera system) and using a technique for distance measurement of the first point. The three-dimensional coordinates (x1, y1, z1) may be determined by installing in the first imaging device 30a, a function of distance measurement of the first point with a single lens.

The three-dimensional coordinates of the first point is defined in a three-dimensional space that includes the first imaging device 30a and the living body 10. The z axis of the three-dimensional space is defined so as to overlap the optical axis of the first imaging device 30a, and the z axis of the three-dimensional space is defined so as to intersect a first plane including the first point at right angles. The origin of the three-dimensional space may be the focal point of the first imaging device 30a.

The first amount of rotation may be determined by using x1 and z1. The second amount of rotation may be determined by using y1 and z1.

Step S105′ (a Process that is an Alternative to Step S105)

The processing device 50 causes the motor-driven device 40 to perform panning rotation by the first amount of rotation, and the processing device 50 causes the motor-driven device 40 to perform tilting rotation by the second amount of rotation Accordingly, the orientation of the first imaging device 30a and the orientation of the second imaging device 30b change synchronously. That is, an angle, in an x-axis direction, made by the optical axis of the first imaging device 30a and the optical axis of the second imaging device 30b, an angle, in a y-axis direction, made by the optical axis of the first imaging device 30a and the optical axis of the second imaging device 30b, and an angle, in a z-axis direction, made by the optical axis of the first imaging device 30a and the optical axis of the second imaging device 30b do not change in response to panning rotation of the motor-driven device 40. The angle, in the x-axis direction, made by the optical axis of the first imaging device 30a and the optical axis of the second imaging device 30b, the angle, in the y-axis direction, made by the optical axis of the first imaging device 30a and the optical axis of the second imaging device 30b, and the angle, in the z-axis direction, made by the optical axis of the first imaging device 30a and the optical axis of the second imaging device 30b do not change in response to tilting rotation of the motor-driven device 40.

### Other Matter 2

The present disclosure is not limited to the above-described embodiment. An embodiment obtained by making various modifications conceived by a person skilled in the art to the present embodiment and a form formed of a combination of constituent elements in different embodiments are also included in the scope of the present disclosure without departing from the gist of the present disclosure.

The imaging system in the present disclosure can obtain biological information of a target portion of a living body. The imaging system in the present disclosure is useful in, for example, biological sensing.

What is claimed is:

1. An imaging system comprising:
a first imaging device that has a first field of view;
a second imaging device that has a second field of view narrower than the first field of view; and
a motor-driven device that is capable of changing an orientation of the second imaging device, wherein
the first imaging device images a living body and generates first image data, the second imaging device images a target portion of the living body and generates second image data,
the second image data is sent to a processing device that generates, on the basis of the second image data, data indicating biological information of the target portion, and
the motor-driven device changes the orientation of the second imaging device on the basis of a position of the living body in an image based on the first image data and maintains a state in which the target portion is included in the second field of view.

2. The imaging system according to claim 1, wherein
the motor-driven device
is capable of changing an orientation of the first imaging device, and
changes the orientation of the first imaging device and the orientation of the second imaging device synchronously on the basis of the position of the living body in the image based on the first image data.

3. The imaging system according to claim 2, wherein
the imaging system includes the processing device.

4. The imaging system according to claim 3, wherein
the image based on the first image data includes a face of the living body, and
the processing device causes the motor-driven device to change the orientation of the first imaging device such that a specific position of the image based on the first image data is included in a region of the face of the living body.

5. The imaging system according to claim 4, wherein
the processing device causes the motor-driven device to change the orientation of the first imaging device and subsequently causes the motor-driven device to further change the orientation of the first imaging device so as to decrease an amount of displacement between the specific position of the image based on the first image data and a specific position of the face of the living body.

6. The imaging system according to claim 2, wherein
the target portion includes a forehead of the living body, and
the processing device causes the motor-driven device to change the orientation of the second imaging device such that the second field of view includes the forehead and eyebrows of the living body.

7. The imaging system according to claim 2, wherein
the processing device causes the motor-driven device to change the orientation of the second imaging device such that the second field of view includes the target portion, and subsequently determines a pixel region of a portion corresponding to the target portion in an image based on the second image data.

8. The imaging system according to claim 7, wherein
the pixel region coincides with a pixel region of a portion corresponding to the target portion in an image based on the second image data before movement of the living body.

9. The imaging system according to claim 1, wherein
the biological information is cerebral blood flow information of the living body.

10. The imaging system according to claim 1, comprising:
at least one light source that emits a light pulse for irradiating the target portion of the living body.

11. A processing device to be used in an imaging system,
the imaging system including:
a first imaging device that has a first field of view,
a second imaging device that has a second field of view narrower than the first field of view, and
a motor-driven device that is capable of changing an orientation of the second imaging device,
the processing device comprising:
a processor; and
a memory that stores a computer program to be executed by the processor, wherein
the computer program causes the processor to
cause the first imaging device to image a living body and to generate first image data,
cause the motor-driven device to change the orientation of the second imaging device on the basis of a position of the living body in an image based on the first image data and to maintain a state in which a target portion of the living body is included in the second field of view,
cause the second imaging device to image the target portion and to generate second image data, and
generate data indicating biological information of the target portion on the basis of the second image data.

12. The processing device according to claim 11, wherein
the motor-driven device is capable of changing an orientation of the first imaging device, and
changing the orientation of the second imaging device on the basis of the position of the living body in the image based on the first image data includes changing the orientation of the first imaging device and the orientation of the second imaging device synchronously on the basis of the position of the living body in the image based on the first image data.

13. A method to be performed by a computer in an imaging system,
the imaging system including:
a first imaging device that has a first field of view,
a second imaging device that has a second field of view narrower than the first field of view, and
a motor-driven device that is capable of changing an orientation of the second imaging device,
the method comprising:
causing the first imaging device to image a living body and to generate first image data;
causing the motor-driven device to change the orientation of the second imaging device on the basis of a position of the living body in an image based on the first image data and to maintain a state in which a target portion of the living body is included in the second field of view;
causing the second imaging device to image the target portion and to generate second image data; and
generating data indicating biological information of the target portion on the basis of the second image data.

14. The method according to claim 13, wherein
the motor-driven device is capable of changing an orientation of the first imaging device, and
changing the orientation of the second imaging device on the basis of the position of the living body in the image based on the first image data includes changing the orientation of the first imaging device and the orientation of the second imaging device synchronously on the basis of the position of the living body in the image based on the first image data.
